SEQUENCE LISTING (30 sequences)

SEQ ID NO: 1 - DNA, 1062 bp, Homo sapiens
atggcccacttcccagggtttggacagagtcttcttttcggatacccagtctacgtgtttggagactgtgtacaaggcgactggtgccccatctctgggggactatgttcggcccgcctacatcgtcacgccctactggccacctgtccagagcatcagatcacctgggaccccatcgatggacgcgttatcggctcagctctacagttccttatccctcgactcccctccttccccacccagagaacctctaagacccttaaggtccttaccccgccaatcactcatacaacccccaacattccaccctccttcctccaggccatgcgcaaatactcccccttccgaaatggatacatggaacccacccttgggcagcacctcccaaccctgtcttttccagaccccggactccggccccaaaacctgtacaccctctggggaggctccgttgtctgcatgtacctctaccagctttccccccccatcacctggcccctcctgccccatgtgattttttgccaccccggccagctcggggccttcctcaccaatgttccctacaaacgaatagaaaaactcctctataaaatttcccttaccacaggggccctaataattctacccgaggactgtttgcccaccacccttttccagcctgctagggcacccgtcacgctgacagcctggcaaaacggcctccttccgttccactcaaccctcaccactccaggccttatttggacatttaccgatggcacgcctatgatttccgggccctgccctaaagatggccagccatctttagtactacagtcctcctcctttatatttcacaaatttcaaaccaaggcctaccacccctcatttctactctcacacggcctcatacagtactcttcctttcataatttgcatctcctatttgaagaatacaccaacatccccatttctctactttttaacgaaaaagaggcagatgacaatgaccatgagccccaaatatcccccgggggcttagagcctctcagtgaaaaacatttccgtgaaacagaagtctga

SEQ ID NO: 2 - protein, 353 aa, Homo sapiens
MAHFPGFGQSLLFGYPVYVFGDCVQGDWCPISGGLCSARLHRHALLATCPEHQITWDPIDGRVIGSALQFLIPRLPSFPTQRTSKTLKVLTPPITHTTPNIPPSFLQAMRKYSPFRNGYMEPTLGQHLPTLSFPDPGLRPQNLYTLWGGSVVCMYLYQLSPPITWPLLPHVIFCHPGQLGAFLTNVPYKRIEKLLYKISLTTGALIILPEDCLPTTLFQPARAPVTLTAWQNGLLPFHSTLTTPGLIWTFTDGTPMISGPCPKDGQPSLVLQSSSFIFHKFQTKAYHPSFLLSHGLIQYSSFHNLHLLFEEYTNIPISLLFNEKEADDNDHEPQISPGGLEPLSEKHFRETEV

SEQ ID NO: 3 - protein, 353 aa, Homo sapiens
MAHFPGFGQSLLFGYPVYVFGDCVQGDWCPISGGLCSARLHRHALLATCPEHQITWDPIDGRVIGSALQFLIPRLPSFPTQRTSKTLKVLTPPITHTTPNIPPSFLQAMRKYSPFRNGYMEPTLGQHLPTLSFPDPASRPQNLYTLWGGSVVCMYLYQLSPPITWPLLPHVIFCHPGQLGAFLTNVPYKRIEKLLYKISLTTGALIILPEDCLPTTLFQPARAPVTLTAWQNGLLPFHSTLTTPGLIWTFTDGTPMISGPCPKDGQPSLVLQSSSFIFHKFQTKAYHPSFLLSHGLIQYSSFHNLHLLFEEYTNIPISLLFNEKEADDNDHEPQISPGGLEPLSEKHFRETEV

SEQ ID NO: 4 - protein, 353 aa, Homo sapiens
MAHFPGFGQSLLFGYPVYVFGDCVQGDWCPISGGLCSARLHRHALLATCPEHQITWDPIDGRVIGSALQFLIPRLPSFPTQRTSKTLKVLTPPITHTTPNIPPSFLQAMRKYSPFRNGYMEPTLGQHLPTLSFPDPGLRPQNLYTLWGGSVVCMYLYQLSPPITWPLLPHVIFCHPGQLGAFLTNVPYKRIEKLLYKISLTTGALIILPEDCLPTTLFQPARAPVTLTAWQNGLLPFHSTLTTPGLIWTFTDGTPMISGPCPKDGQPSLVLQSSSFIFHKFQTKAYHPSFLLSHGLIQYSSFHNLHLLFEEYTNIPISRSFNEKEADDNDHEPQISPGGLEPLSEKHFRETEV

SEQ ID NO: 5 - DNA, 618 bp, Homo sapiens
atggctgctagtggactgttccgatgcctgcctgtgagttgccctgaggacctgctggtggaggagctggtggatggcctgctgagcctggaggaggagctgaaggacaaggaggaggagaaggccgtgctggatggcctgctgagcctggaggaggagtcccgcggccggctgaggagaggaccacctggcgagaaggccccacccagaggcgagacacacagggacagacagaggagggcagaggagaagaggaagcggaagaaagagcgcgagaaggaggaggagaagcagatcgccgagtacctgaagcggaaggaagaggagaaggccagaaggaggaggcgggcagagaagaaggcagcagacgtggccagaaggaagcaggaggagcaggagagaagggagcggaagtggcgccagggagcagagaaggcaaagcagcactctgccagaaaggagaagatgcaggagctgggcatcgatggctatacacggcagctggagggagaggtggagagcctggaggcagagagaaggaagctgctgcaggagaaggaggatttgatgggggaggtcaactactggcaggggaggctggaggccatgtggctgcag

SEQ ID NO: 6 - protein, 206 aa, Homo sapiens
MAASGLFRCLPVSCPEDLLVEELVDGLLSLEEELKDKEEEKAVLDGLLSLEEESRGRLRRGPPGEKAPPRGETHRDRQRRAEEKRKRKKEREKEEEKQIAEYLKRKEEEKARRRRRAEKKAADVARRKQEEQERRERKWRQGAEKAKQHSARKEKMQELGIDGYTRQLEGEVESLEAERRKLLQEKEDLMGEVNYWQGRLEAMWLQ

SEQ ID NO: 7 - DNA, 618 bp, Artificial Sequence
  Synthetic polynucleotide
atggctgctagtggactgttccgatgcctgcctgtgagttgccctgaggacctgctggtggaggagctggtggatggcgcagcagccgctgcggcggagctgaaggacaaggaggaggagaaggccgtgctggatggcctgctgagcctggaggaggagtcccgcggccggctgaggagaggaccacctggcgagaaggccccacccagaggcgagacacacagggacagacagaggagggcagaggagaagaggaagcggaagaaagagcgcgagaaggaggaggagaagcagatcgccgagtacctgaagcggaaggaagaggagaaggccagaaggaggaggcgggcagagaagaaggcagcagacgtggccagaaggaagcaggaggagcaggagagaagggagcggaagtggcgccagggagcagagaaggcaaagcagcactctgccagaaaggagaagatgcaggagctgggcatcgatggctatacacggcagctggagggagaggtggagagcctggaggcagagagaaggaagctgctgcaggagaaggaggatttgatgggggaggtcaactactggcaggggaggctggaggccatgtggctgcag

SEQ ID NO: 8 - protein, 206 aa, Artificial Sequence
  Synthetic polypeptide
MAASGLFRCLPVSCPEDLLVEELVDGAAAAAAELKDKEEEKAVLDGLLSLEEESRGRLRRGPPGEKAPPRGETHRDRQRRAEEKRKRKKEREKEEEKQIAEYLKRKEEEKARRRRRAEKKAADVARRKQEEQERRERKWRQGAEKAKQHSARKEKMQELGIDGYTRQLEGEVESLEAERRKLLQEKEDLMGEVNYWQGRLEAMWLQ

SEQ ID NO: 9 - DNA, 618 bp, Artificial Sequence
  Synthetic polynucleotide
atggctgctagtggactgttccgatgcctgcctgtgagttgccctgaggacctgctggtggaggagctggtggatggcctgctgagcctggaggaggagctgaaggacaaggaggaggagaaggccgtgctggatggcctgctgagcctggaggaggagtcccgcggccggctgaggagaggaccacctggcgagaaggccccacccagaggcgagacacacagggacagacagaggagggcagaggagaagaggaagcggaagaaagagcgcgagaaggaggaggagaagcagatcgccgagtacctgaagcggaaggaagaggagaaggccagaaggaggaggcgggcagagaagaaggcagcagacgcggccgcagcggcagcggaggagcaggagagaagggagcggaagtggcgccagggagcagagaaggcaaagcagcactctgccagaaaggagaagatgcaggagctgggcatcgatggctatacacggcagctggagggagaggtggagagcctggaggcagagagaaggaagctgctgcaggagaaggaggatttgatgggggaggtcaactactggcaggggaggctggaggccatgtggctgcag

SEQ ID NO: 10 - protein, 618 aa, Artificial Sequence
  Synthetic polypeptide
ATGGCTGCTAGTGGACTGTTCCGATGCCTGCCTGTGAGTTGCCCTGAGGACCTGCTGGTGGAGGAGCTGGTGGATGGCCTGCTGAGCCTGGAGGAGGAGCTGAAGGACAAGGAGGAGGAGAAGGCCGTGCTGGATGGCCTGCTGAGCCTGGAGGAGGAGTCCCGCGGCCGGCTGAGGAGAGGACCACCTGGCGAGAAGGCCCCACCCAGAGGCGAGACACACAGGGACAGACAGAGGAGGGCAGAGGAGAAGAGGAAGCGGAAGAAAGAGCGCGAGAAGGAGGAGGAGAAGCAGATCGCCGAGTACCTGAAGCGGAAGGAAGAGGAGAAGGCCAGAAGGAGGAGGCGGGCAGAGAAGAAGGCAGCAGACGCGGCCGCAGCGGCAGCGGAGGAGCAGGAGAGAAGGGAGCGGAAGTGGCGCCAGGGAGCAGAGAAGGCAAAGCAGCACTCTGCCAGAAAGGAGAAGATGCAGGAGCTGGGCATCGATGGCTATACACGGCAGCTGGAGGGAGAGGTGGAGAGCCTGGAGGCAGAGAGAAGGAAGCTGCTGCAGGAGAAGGAGGATTTGATGGGGGAGGTCAACTACTGGCAGGGGAGGCTGGAGGCCATGTGGCTGCAG

SEQ ID NO: 11 - DNA, 618 bp, Artificial Sequence
  Synthetic polynucleotide
atggctgctagtggactgttccgatgcctgcctgtgagttgccctgaggacctgctggtggaggagctggtggatggcctgctgagcctggaggaggagctgaaggacaaggaggaggagaaggccgtgctggatggcctgctgagcctggaggaggagtcccgcggccggctgaggagaggaccacctggcgagaaggccccacccagaggcgaggcagccgcggccagacagaggagggcagaggagaagaggaagcggaagaaagagcgcgagaaggaggaggagaagcagatcgccgagtacctgaagcggaaggaagaggagaaggccagaaggaggaggcgggcagagaagaaggcagcagacgtggccagaaggaagcaggaggagcaggagagaagggagcggaagtggcgccagggagcagagaaggcaaagcagcactctgccagaaaggagaagatgcaggagctgggcatcgatggctatacacggcagctggagggagaggtggagagcctggaggcagagagaaggaagctgctgcaggagaaggaggatttgatgggggaggtcaactactggcaggggaggctggaggccatgtggctgcag

SEQ ID NO: 12 - protein, 206 aa, Artificial Sequence
  Synthetic polypeptide
MAASGLFRCLPVSCPEDLLVEELVDGLLSLEEELKDKEEEKAVLDGLLSLEEESRGRLRRGPPGEKAPPRGEAAAARQRRAEEKRKRKKEREKEEEKQIAEYLKRKEEEKARRRRRAEKKAADVARRKQEEQERRERKWRQGAEKAKQHSARKEKMQELGIDGYTRQLEGEVESLEAERRKLLQEKEDLMGEVNYWQGRLEAMWLQ

SEQ ID NO: 13 - DNA, 618 bp, Artificial Sequence
  Synthetic polynucleotide
atggctgctagtggactgttccgatgcctgcctgtgagttgccctgaggacctgctggtggaggagctggtggatggcctgctgagcctggaggaggagctgaaggacaaggaggaggagaaggccgtgctggatggcctgctgagcctggaggaggagtcccgcggccggctgaggagaggaccacctggcgagaaggccccacccagaggcgagacacacagggacagacagaggagggcagaggagaagaggaagcggaagaaagagcgcgagaaggaggaggagaagcagatcgccgagtacctgaagcggaaggaagaggagaaggccagaaggaggaggcgggcagagaagaaggcagcagacgtggccagaaggaagcaggaggagcaggagagaagggagcggaagtggcgccagggagcagagaaggcaaagcagcactctgccagaaaggagaagatgcaggagctgggcatcgatggctatacacggcagctggagggagaggtggagagcctggaggcagagagagcggcagcggctgcagcgaaggaggatttgatgggggaggtcaactactggcaggggaggctggaggccatgtggctgcag

SEQ ID NO: 14 - protein, 206 aa, Artificial Sequence
  Synthetic polypeptide
MAASGLFRCLPVSCPEDLLVEELVDGLLSLEEELKDKEEEKAVLDGLLSLEEESRGRLRRGPPGEKAPPRGETHRDRQRRAEEKRKRKKEREKEEEKQIAEYLKRKEEEKARRRRRAEKKAADVARRKQEEQERRERKWRQGAEKAKQHSARKEKMQELGIDGYTRQLEGEVESLEAERAAAAAAKEDLMGEVNYWQGRLEAMWLQ

SEQ ID NO: 15 - DNA, 618 bp, Artificial Sequence
  Synthetic polynucleotide
atggctgctagtggactgttccgatgcctgcctgtgagttgccctgaggacctgctggtggaggagctggtggatggcctgctgagcctggaggaggagctgaaggacaaggaggaggagaaggccgtgctggatggcctgctgagcctggaggaggagtcccgcggccggctgaggagaggaccacctggcgagaaggccccacccagaggcgagacacacagggacagacagaggagggcagaggagaagaggaagcggaagaaagagcgcgagaaggaggaggagaagcagatcgccgagtacctgaagcggaaggaagaggagaaggccagaaggagggctgcggcagcggcagcggcagcagacgtggccagaaggaagcaggaggagcaggagagaagggagcggaagtggcgccagggagcagagaaggcaaagcagcactctgccagaaaggagaagatgcaggagctgggcatcgatggctatacacggcagctggagggagaggtggagagcctggaggcagagagaaggaagctgctgcaggagaaggaggatttgatgggggaggtcaactactggcaggggaggctggaggccatgtggctgcag

SEQ ID NO: 16 - protein, 206 aa, Artificial Sequence
  Synthetic polypeptide
MAASGLFRCLPVSCPEDLLVEELVDGLLSLEEELKDKEEEKAVLDGLLSLEEESRGRLRRGPPGEKAPPRGETHRDRQRRAEEKRKRKKEREKEEEKQIAEYLKRKEEEKARRRAAAAAAAADVARRKQEEQERRERKWRQGAEKAKQHSARKEKMQELGIDGYTRQLEGEVESLEAERRKLLQEKEDLMGEVNYWQGRLEAMWLQ

SEQ ID NO: 17 - DNA, 1062 bp, Artificial Sequence
  Synthetic polynucleotide
atggcccacttcccagggtttggacagagtcttcttttcggatacccagtctacgtgtttggagactgtgtacaaggcgactggtgccccatctctgggggactatgttcggcccgcctacatcgtcacgccctactggccacctgtccagagcatcagatcacctgggaccccatcgatggacgcgttatcggctcagctctacagttccttatccctcgactcccctccttccccacccagagaacctctaagacccttaaggtccttaccccgccaatcactcatacaacccccaacattccaccctccttcctccaggccatgcgcaaatactcccccttccgaaatggatacatggaacccacccttgggcagcacctcccaaccctgtcttttccagaccccggactccggccccaaaacctgtacaccctctggggaggctccgttgtctgcatgtacctctaccagctttccccccccatcacctggcccctcctgccccatgtgattttttgccaccccggccagctcggggccttcctcaccaatgttccctacaaacgaatagaaaaactcctctataaaatttcccttaccacaggggccctaataattctacccgaggactgtttgcccaccacccttttccagcctgctagggcacccgtcacgctgacagcctggcaaaacggcctccttccgttccactcaaccctcaccactccaggccttatttggacatttaccgatggcacgcctatgatttccgggccctgccctaaagatggccagccatctttagtactacagtcctcctcctttatatttcacaaatttcaaaccaaggcctaccacccctcatttctactctcacacggcctcatacagtactcttcctttcataatttgcatctcctatttgaagaatacaccaacatccccgctgctgcagctgctgccgaaaaagaggcagatgacaatgaccatgagccccaaatatcccccgggggcttagagcctctcagtgaaaaacatttccgtgaaacagaagtctga

SEQ ID NO: 18 - protein, 353 aa, Artificial Sequence
  Synthetic polypeptide
MAHFPGFGQSLLFGYPVYVFGDCVQGDWCPISGGLCSARLHRHALLATCPEHQITWDPIDGRVIGSALQFLIPRLPSFPTQRTSKTLKVLTPPITHTTPNIPPSFLQAMRKYSPFRNGYMEPTLGQHLPTLSFPDPGLRPQNLYTLWGGSVVCMYLYQLSPPITWPLLPHVIFCHPGQLGAFLTNVPYKRIEKLLYKISLTTGALIILPEDCLPTTLFQPARAPVTLTAWQNGLLPFHSTLTTPGLIWTFTDGTPMISGPCPKDGQPSLVLQSSSFIFHKFQTKAYHPSFLLSHGLIQYSSFHNLHLLFEEYTNIPAAAAAAEKEADDNDHEPQISPGGLEPLSEKHFRETEV

SEQ ID NO: 19 - DNA, 618 bp, Artificial Sequence; identical to SEQ ID NO: 7
  Synthetic polynucleotide

SEQ ID NO: 20 - protein, 206 aa, Artificial Sequence; identical to SEQ ID NO: 8
  Synthetic polypeptide

SEQ ID NO: 21 - DNA, 1686 bp, Artificial Sequence
  Synthetic polynucleotide
atggcccacttcccagggtttggacagagtcttcttttcggatacccagtctacgtgtttggagactgtgtacaaggcgactggtgccccatctctgggggactatgttcggcccgcctacatcgtcacgccctactggccacctgtccagagcatcagatcacctgggaccccatcgatggacgcgttatcggctcagctctacagttccttatccctcgactcccctccttccccacccagagaacctctaagacccttaaggtccttaccccgccaatcactcatacaacccccaacattccaccctccttcctccaggccatgcgcaaatactcccccttccgaaatggatacatggaacccacccttgggcagcacctcccaaccctgtcttttccagaccccggactccggccccaaaacctgtacaccctctggggaggctccgttgtctgcatgtacctctaccagctttccccccccatcacctggcccctcctgccccatgtgattttttgccaccccggccagctcggggccttcctcaccaatgttccctacaaacgaatagaaaaactcctctataaaatttcccttaccacaggggccctaataattctacccgaggactgtttgcccaccacccttttccagcctgctagggcacccgtcacgctgacagcctggcaaaacggcctccttccgttccactcaaccctcaccactccaggccttatttggacatttaccgatggcacgcctatgatttccgggccctgccctaaagatggccagccatctttagtactacagtcctcctcctttatatttcacaaatttcaaaccaaggcctaccacccctcatttctactctcacacggcctcatacagtactcttcctttcataatttgcatctcctatttgaagaatacaccaacatccccatttctctactttttaacgaaaaagaggcagatgacaatgaccatgagccccaaatatcccccgggggcttagagcctctcagtgaaaaacatttccgtgaaacagaagtcatggcggcctcagggctgtttcgatgcttgcctgtgtcatgcccggaggacctgctggtggaggaattggtggacgggctattatccttggaggaagagttaaaggacaaggaggaggagaaagctgtgcttgacggtttgctatccttagaagaggaaagccgcggccggctgcgacggggccctccaggggagaaagcgccacctcgcggggaaacgcatcgtgatcggcagcgacgggctgaggagaagaggaagcgaaaaaaagagcgggagaaagaggaggaaaagcagattgctgagtatttgaaaaggaaggaagaggagaaggcacggcgcaggaggcgggcggagaagaaggccgctgacgtcgccaggaggaagcaggaagagcaggagcgccgtgagcgcaagtggagacaaggggctgagaaggcgaaacagcatagtgctaggaaagaaaaaatgcaggagttggggattgatggctatactagacagttggaaggcgaggtggagtccttggaggctgaacggaggaagttgctgcaggagaaggaggatttgatgggagaggttaattattggcaggggaggctggaggcgatgtggttgcaataagctagc

SEQ ID NO: 22 - protein, 559 aa, Artificial Sequence
  Synthetic polypeptide
MAHFPGFGQSLLFGYPVYVFGDCVQGDWCPISGGLCSARLHRHALLATCPEHQITWDPIDGRVIGSALQFLIPRLPSFPTQRTSKTLKVLTPPITHTTPNIPPSFLQAMRKYSPFRNGYMEPTLGQHLPTLSFPDPGLRPQNLYTLWGGSVVCMYLYQLSPPITWPLLPHVIFCHPGQLGAFLTNVPYKRIEKLLYKISLTTGALIILPEDCLPTTLFQPARAPVTLTAWQNGLLPFHSTLTTPGLIWTFTDGTPMISGPCPKDGQPSLVLQSSSFIFHKFQTKAYHPSFLLSHGLIQYSSFHNLHLLFEEYTNIPISLLFNEKEADDNDHEPQISPGGLEPLSEKHFRETEVMAASGLFRCLPVSCPEDLLVEELVDGLLSLEEELKDKEEEKAVLDGLLSLEEESRGRLRRGPPGEKAPPRGETHRDRQRRAEEKRKRKKEREKEEEKQIAEYLKRKEEEKARRRRRAEKKAADVARRKQEEQERRERKWRQGAEKAKQHSARKEKMQELGIDGYTRQLEGEVESLEAERRKLLQEKEDLMGEVNYWQGRLEAMWLQ

SEQ ID NO: 23 - DNA, 1686 bp, Artificial Sequence
  Synthetic polynucleotide
atggcccacttcccagggtttggacagagtcttcttttcggatacccagtctacgtgtttggagactgtgtacaaggcgactggtgccccatctctgggggactatgttcggcccgcctacatcgtcacgccctactggccacctgtccagagcatcagatcacctgggaccccatcgatggacgcgttatcggctcagctctacagttccttatccctcgactcccctccttccccacccagagaacctctaagacccttaaggtccttaccccgccaatcactcatacaacccccaacattccaccctccttcctccaggccatgcgcaaatactcccccttccgaaatggatacatggaacccacccttgggcagcacctcccaaccctgtcttttccagaccccggactccggccccaaaacctgtacaccctctggggaggctccgttgtctgcatgtacctctaccagctttccccccccatcacctggcccctcctgccccatgtgattttttgccaccccggccagctcggggccttcctcaccaatgttccctacaaacgaatagaaaaactcctctataaaatttcccttaccacaggggccctaataattctacccgaggactgtttgcccaccacccttttccagcctgctagggcacccgtcacgctgacagcctggcaaaacggcctccttccgttccactcaaccctcaccactccaggccttatttggacatttaccgatggcacgcctatgatttccgggccctgccctaaagatggccagccatctttagtactacagtcctcctcctttatatttcacaaatttcaaaccaaggcctaccacccctcatttctactctcacacggcctcatacagtactcttcctttcataatttgcatctcctatttgaagaatacaccaacatccccgctgctgcagctgctgccgaaaaagaggcagatgacaatgaccatgagccccaaatatcccccgggggcttagagcctctcagtgaaaaacatttccgtgaaacagaagtcatggcggcctcagggctgtttcgatgcttgcctgtgtcatgcccggaggacctgctggtggaggaattggtggacggggcagcagccgctgcggcggagttaaaggacaaggaggaggagaaagctgtgcttgacggtttgctatccttagaagaggaaagccgcggccggctgcgacggggccctccaggggagaaagcgccacctcgcggggaaacgcatcgtgatcggcagcgacgggctgaggagaagaggaagcgaaaaaaagagcgggagaaagaggaggaaaagcagattgctgagtatttgaaaaggaaggaagaggagaaggcacggcgcaggaggcgggcggagaagaaggccgctgacgtcgccaggaggaagcaggaagagcaggagcgccgtgagcgcaagtggagacaaggggctgagaaggcgaaacagcatagtgctaggaaagaaaaaatgcaggagttggggattgatggctatactagacagttggaaggcgaggtggagtccttggaggctgaacggaggaagttgctgcaggagaaggaggatttgatgggagaggttaattattggcaggggaggctggaggcgatgtggttgcaataagctagc

SEQ ID NO: 24 - protein, 559 aa, Artificial Sequence
  Synthetic polypeptide
MAHFPGFGQSLLFGYPVYVFGDCVQGDWCPISGGLCSARLHRHALLATCPEHQITWDPIDGRVIGSALQFLIPRLPSFPTQRTSKTLKVLTPPITHTTPNIPPSFLQAMRKYSPFRNGYMEPTLGQHLPTLSFPDPGLRPQNLYTLWGGSVVCMYLYQLSPPITWPLLPHVIFCHPGQLGAFLTNVPYKRIEKLLYKISLTTGALIILPEDCLPTTLFQPARAPVTLTAWQNGLLPFHSTLTTPGLIWTFTDGTPMISGPCPKDGQPSLVLQSSSFIFHKFQTKAYHPSFLLSHGLIQYSSFHNLHLLFEEYTNIPAAAAAAEKEADDNDHEPQISPGGLEPLSEKHFRETEVMAASGLFRCLPVSCPEDLLVEELVDGAAAAAAELKDKEEEKAVLDGLLSLEEESRGRLRRGPPGEKAPPRGETHRDRQRRAEEKRKRKKEREKEEEKQIAEYLKRKEEEKARRRRRAEKKAADVARRKQEEQERRERKWRQGAEKAKQHSARKEKMQELGIDGYTRQLEGEVESLEAERRKLLQEKEDLMGEVNYWQGRLEAMWLQ

SEQ ID NO: 25 - DNA, 1686 bp, Artificial Sequence
  Synthetic polynucleotide
atggcggcctcagggctgtttcgatgcttgcctgtgtcatgcccggaggacctgctggtggaggaattggtggacgggctattatccttggaggaagagttaaaggacaaggaggaggagaaagctgtgcttgacggtttgctatccttagaagaggaaagccgcggccggctgcgacggggccctccaggggagaaagcgccacctcgcggggaaacgcatcgtgatcggcagcgacgggctgaggagaagaggaagcgaaaaaaagagcgggagaaagaggaggaaaagcagattgctgagtatttgaaaaggaaggaagaggagaaggcacggcgcaggaggcgggcggagaagaaggccgctgacgtcgccaggaggaagcaggaagagcaggagcgccgtgagcgcaagtggagacaaggggctgagaaggcgaaacagcatagtgctaggaaagaaaaaatgcaggagttggggattgatggctatactagacagttggaaggcgaggtggagtccttggaggctgaacggaggaagttgctgcaggagaaggaggatttgatgggagaggttaattattggcaggggaggctggaggcgatgtggttgcaaatggcccacttcccagggtttggacagagtcttcttttcggatacccagtctacgtgtttggagactgtgtacaaggcgactggtgccccatctctgggggactatgttcggcccgcctacatcgtcacgccctactggccacctgtccagagcatcagatcacctgggaccccatcgatggacgcgttatcggctcagctctacagttccttatccctcgactcccctccttccccacccagagaacctctaagacccttaaggtccttaccccgccaatcactcatacaacccccaacattccaccctccttcctccaggccatgcgcaaatactcccccttccgaaatggatacatggaacccacccttgggcagcacctcccaaccctgtcttttccagaccccggactccggccccaaaacctgtacaccctctggggaggctccgttgtctgcatgtacctctaccagctttccccccccatcacctggcccctcctgccccatgtgattttttgccaccccggccagctcggggccttcctcaccaatgttccctacaaacgaatagaaaaactcctctataaaatttcccttaccacaggggccctaataattctacccgaggactgtttgcccaccacccttttccagcctgctagggcacccgtcacgctgacagcctggcaaaacggcctccttccgttccactcaaccctcaccactccaggccttatttggacatttaccgatggcacgcctatgatttccgggccctgccctaaagatggccagccatctttagtactacagtcctcctcctttatatttcacaaatttcaaaccaaggcctaccacccctcatttctactctcacacggcctcatacagtactcttcctttcataatttgcatctcctatttgaagaatacaccaacatccccatttctctactttttaacgaaaaagaggcagatgacaatgaccatgagccccaaatatcccccgggggcttagagcctctcagtgaaaaacatttccgtgaaacagaagtctgagctagc

SEQ ID NO: 26 - protein, 559 aa, Artificial Sequence
  Synthetic polypeptide
MAASGLFRCLPVSCPEDLLVEELVDGLLSLEEELKDKEEEKAVLDGLLSLEEESRGRLRRGPPGEKAPPRGETHRDRQRRAEEKRKRKKEREKEEEKQIAEYLKRKEEEKARRRRRAEKKAADVARRKQEEQERRERKWRQGAEKAKQHSARKEKMQELGIDGYTRQLEGEVESLEAERRKLLQEKEDLMGEVNYWQGRLEAMWLQMAHFPGFGQSLLFGYPVYVFGDCVQGDWCPISGGLCSARLHRHALLATCPEHQITWDPIDGRVIGSALQFLIPRLPSFPTQRTSKTLKVLTPPITHTTPNIPPSFLQAMRKYSPFRNGYMEPTLGQHLPTLSFPDPGLRPQNLYTLWGGSVVCMYLYQLSPPITWPLLPHVIFCHPGQLGAFLTNVPYKRIEKLLYKISLTTGALIILPEDCLPTTLFQPARAPVTLTAWQNGLLPFHSTLTTPGLIWTFTDGTPMISGPCPKDGQPSLVLQSSSFIFHKFQTKAYHPSFLLSHGLIQYSSFHNLHLLFEEYTNIPISLLFNEKEADDNDHEPQISPGGLEPLSEKHFRETEV

SEQ ID NO: 27 - DNA, 1686 bp, Artificial Sequence
  Synthetic polynucleotide
atggcggcctcagggctgtttcgatgcttgcctgtgtcatgcccggaggacctgctggtggaggaattggtggacggggcagcagccgctgcggcggagttaaaggacaaggaggaggagaaagctgtgcttgacggtttgctatccttagaagaggaaagccgcggccggctgcgacggggccctccaggggagaaagcgccacctcgcggggaaacgcatcgtgatcggcagcgacgggctgaggagaagaggaagcgaaaaaaagagcgggagaaagaggaggaaaagcagattgctgagtatttgaaaaggaaggaagaggagaaggcacggcgcaggaggcgggcggagaagaaggccgctgacgtcgccaggaggaagcaggaagagcaggagcgccgtgagcgcaagtggagacaaggggctgagaaggcgaaacagcatagtgctaggaaagaaaaaatgcaggagttggggattgatggctatactagacagttggaaggcgaggtggagtccttggaggctgaacggaggaagttgctgcaggagaaggaggatttgatgggagaggttaattattggcaggggaggctggaggcgatgtggttgcaaatggcccacttcccagggtttggacagagtcttcttttcggatacccagtctacgtgtttggagactgtgtacaaggcgactggtgccccatctctgggggactatgttcggcccgcctacatcgtcacgccctactggccacctgtccagagcatcagatcacctgggaccccatcgatggacgcgttatcggctcagctctacagttccttatccctcgactcccctccttccccacccagagaacctctaagacccttaaggtccttaccccgccaatcactcatacaacccccaacattccaccctccttcctccaggccatgcgcaaatactcccccttccgaaatggatacatggaacccacccttgggcagcacctcccaaccctgtcttttccagaccccggactccggccccaaaacctgtacaccctctggggaggctccgttgtctgcatgtacctctaccagctttccccccccatcacctggcccctcctgccccatgtgattttttgccaccccggccagctcggggccttcctcaccaatgttccctacaaacgaatagaaaaactcctctataaaatttcccttaccacaggggccctaataattctacccgaggactgtttgcccaccacccttttccagcctgctagggcacccgtcacgctgacagcctggcaaaacggcctccttccgttccactcaaccctcaccactccaggccttatttggacatttaccgatggcacgcctatgatttccgggccctgccctaaagatggccagccatctttagtactacagtcctcctcctttatatttcacaaatttcaaaccaaggcctaccacccctcatttctactctcacacggcctcatacagtactcttcctttcataatttgcatctcctatttgaagaatacaccaacatccccgctgctgcagctgctgccgaaaaagaggcagatgacaatgaccatgagccccaaatatcccccgggggcttagagcctctcagtgaaaaacatttccgtgaaacagaagtctgagctagc

SEQ ID NO: 28 - protein, 559 aa, Artificial Sequence
  Synthetic polypeptide
MAASGLFRCLPVSCPEDLLVEELVDGAAAAAAELKDKEEEKAVLDGLLSLEEESRGRLRRGPPGEKAPPRGETHRDRQRRAEEKRKRKKEREKEEEKQIAEYLKRKEEEKARRRRRAEKKAADVARRKQEEQERRERKWRQGAEKAKQHSARKEKMQELGIDGYTRQLEGEVESLEAERRKLLQEKEDLMGEVNYWQGRLEAMWLQMAHFPGFGQSLLFGYPVYVFGDCVQGDWCPISGGLCSARLHRHALLATCPEHQITWDPIDGRVIGSALQFLIPRLPSFPTQRTSKTLKVLTPPITHTTPNIPPSFLQAMRKYSPFRNGYMEPTLGQHLPTLSFPDPGLRPQNLYTLWGGSVVCMYLYQLSPPITWPLLPHVIFCHPGQLGAFLTNVPYKRIEKLLYKISLTTGALIILPEDCLPTTLFQPARAPVTLTAWQNGLLPFHSTLTTPGLIWTFTDGTPMISGPCPKDGQPSLVLQSSSFIFHKFQTKAYHPSFLLSHGLIQYSSFHNLHLLFEEYTNIPAAAAAAEKEADDNDHEPQISPGGLEPLSEKHFRETEV

SEQ ID NO: 29 - DNA, 1461 bp, Homo sapiens
atgggtaagtttctcgccactttgattttattcttccagttctgccccctcatcctcggtgattacagccccagctgctgtactctcacaattggagtctcctcataccactctaaaccctgcaatcctgcccagccagtttgttcgtggaccctcgacctgccggccctttcagcagatcaggccctacagcccccctgccctaatctagtaagttactccagctaccatgccacctattccctatatctattccctcattggattaaaaagccaaaccgaaatggcggaggctattattcagcctcttattcagacccttgttccttaaagtgcccatacctggggtgccaatcatggacctgcccctatacaggagccgtctccagcccctactggaagtttcagcaagatgtcaattttactcaagaagtttcacgcctcaatattaatctccatttttcgaaatgcggttttcccttctcccttctagtcgacgctccaggatatgaccccatctggttccttaataccgaacccagccaactgcctcccaccgcccctcctctactcccccactctaacctagaccacatcctggagccctctataccatggaaatcaaaactcctgacccttgtccagttaaccctacaaagcactaattatacttgcattgtctgtatcgatcgtgccagcctatccacttggcacgtcctatactctcccaacgtctctgttccatcctcttcttctacccccctcctttacccatcgttagcgcttccagccccccacctgacgttaccatttaactggacccactgctttgacccccagattcaagctatagtctcctccccttgtcataactccctcatcctgccccccttttccttgtcacctgttcccaccctaggatcccgctcccgccgagcggtaccggtggcggtctggcttgtctccgccctggccatgggagccggggtggctggcgggattaccggctccatgtccctcgcctcaggaaagagcctcttacatgaggtggacaaagatatttcccaattaactcaagcaatagtcaaaaaccacaaaaatctactcaaaattgcgcagtatgctgcccagaacagacgaggccttgatctcctgttctgggagcaaggaggattatgcaaagcattacaagaacagtgctgttttctgaatattactaattcccatgtctcaatactacaagaaagaccccccctggagaatcgagtcctgactggctggggccttaactgggaccttggcctctcacagtgggctagagaggccttacaaactggaatcacccttgtcgcgctactccttcttgttatccttgcaggaccatgcatctacattaaattaaagcacaccaagaaaagacagatttatacagacatagagatgaaccgacttggaaggtaa

SEQ ID NO: 30 - protein, 486 aa, Homo sapiens
MGKFLATLILFFQFCPLILGDYSPSCCTLTIGVSSYHSKPCNPAQPVCSWTLDLPALSADQALQPPCPNLVSYSSYHATYSLYLFPHWIKKPNRNGGGYYSASYSDPCSLKCPYLGCQSWTCPYTGAVSSPYWKFQQDVNFTQEVSRLNINLHFSKCGFPFSLLVDAPGYDPIWFLNTEPSQLPPTAPPLLPHSNLDHILEPSIPWKSKLLTLVQLTLQSTNYTCIVCIDRASLSTWHVLYSPNVSVPSSSSTPLLYPSLALPAPHLTLPFNWTHCFDPQIQAIVSSPCHNSLILPPFSLSPVPTLGSRSRRAVPVAVWLVSALAMGAGVAGGITGSMSLASGKSLLHEVDKDISQLTQAIVKNHKNLLKIAQYAAQNRRGLDLLFWEQGGLCKALQEQCCFLNITNSHVSILQERPPLENRVLTGWGLNWDLGLSQWAREALQTGITLVALLLLVILAGPCIYIKLKHTKKRQIYTDIEMNRLGR

(19) United States
(12) Patent Application Publication
(10) Pub. No.: US 2022/0184202 A1
(43) Pub. Date: Jun. 16, 2022

(54) A RECOMBINANT HTLV-1 VACCINE

(71) Applicant: UNIVERSITY OF MIAMI, MIAMI, FL (US)

(72) Inventor: GLEN N BARBER, MIAMI, FL (US)

(73) Assignee: UNIVERSITY OF MIAMI, MIAMI, FL (US)

(21) Appl. No.: 17/603,331

(22) PCT Filed: Apr. 10, 2020
(86) PCT No.: PCT/US2020/027649

§ 371 Date: Oct. 12, 2021

Related U.S. Application Data
(60) Provisional application No. 62/833,025, filed on Apr. 12, 2019.

(51) Int. Cl. A61K 39/12; A61P 31/14; C07K 14/005; C12N 15/86

(52) U.S. Cl. CPC A61K 39/12; A61P 31/14; A61K 2039/5256; C12N 15/86; C07K 14/005

(57) ABSTRACT

The invention relates to a vector and/or vaccine that can be used for therapeutic and preventive purposes. The virus is based on vesicular stomatitis virus (VSV) with a substituted VSV G (glycoprotein) for HTLV-1 G, referred to as gp62. The vector and/or vaccine further comprise a fusion protein comprising HTLV-1 regulatory proteins (HBZ and TAX) together to make a fusion product (HBZ-TAX) and mutated versions thereof. The vector and/or vaccine do not impede innate immune signaling and generate neutralizing antibodies and CTLs to gp62, HBZ, and TAX.

### PRIORITY CLAIM

This application is the national phase of (1) an International Application PCT/US2020/027649, filed Apr. 10, 2020, which claims the benefit of priority to (2) U.S. provisional application Ser. No. 62/833,025, filed on Apr. 12, 2019, which applications (1)-(2) are hereby expressly incorporated by reference in their entireties and for all purposes.

### STATEMENT REGARDING FEDERALLY SPONSORED RESEARCH

This invention was made with government support under P30AI073961 awarded by the National Institutes of Health. The government has certain rights in the invention.

### REFERENCE TO SEQUENCE LISTING

This application contains a Sequence Listing submitted as an electronic text file named “STBG-01007US1_ST25.txt”, having a size in bytes of 77,824 bytes, created on Oct. 28, 2021. The information contained in this electronic file is hereby incorporated by reference in its entirety pursuant to 37 CFR § 1.52(e)(5).

### BACKGROUND OF THE INVENTION

### Field of the Invention

This disclosure relates generally to immunology and chimeric proteins and fusion proteins. In particular, this disclosure provides and vectors and vaccines for producing protective and therapeutic immune responses to Human T-cell leukemia virus type-1 (HTLV-1).

### Description of Related Art

Human T-cell leukemia virus type-1 (HTLV-1) is a human retrovirus that is the causative agent of a severe form of leukemia known as Adult T cell leukemia (ATL) as well as several inflammatory disorders with the most severe being human myelopathy/tropical spastic paraparesis (HAM/TSP). The HTLV-1 genome is comprised of two copies of ssRNA that is converted to dsDNA which is then added to the host genome known as a provirus. HTLV-1 infection is endemic in many areas around the world including southern Japan, the southern United States, central Australia, the Caribbean, South America, equatorial Africa, and the middle East. The majority of infected carriers are asymptomatic for their lifetime however an estimated 5% of HTLV-1 positive individuals will develop ATL or 2% into HAM/TSP after prolonged latency periods. Despite the relatively low penetrance of HTLV-1 associated diseases, HTLV-1 is a major problem in endemic communities as there are no effective treatment options for either ATL or HAM/TSP afflicted individuals.

Development of either disease requires a rather long latency period in which the virus can persist in the host for extended periods of time while evading the immune system. HTLV-1 is usually transmitted through breastfeeding, sexual contact, or blood transfusion. Once infected HTLV-1 spreads throughout the host by two main mechanisms. The de novo infection of host cells through infectious virions which is relatively inefficient as the cell free virus is poorly infectious and the clonal proliferation of infected cells carrying the HTLV-1 provirus. In HTLV infected individuals the virus is almost entirely cell associated with the virion load being virtually undetectable.

ATL is a highly aggressive malignancy of activated CD4+ T lymphocytes that develops after a long latency period in infected individuals. It manifests clinically into 4 subtypes: (1) smoldering, (2) chronic, (3) acute, and (4) lymphoma. Each subtype is defined according to diagnostic criteria such as lymphadenopathy, splenomegaly, hepatomegaly, hypercalcemia, skin and pulmonary lesions, organ infiltration. The more aggressive subtypes are acute and lymphoma and each carry a very dire prognosis with median survival time of approximately 9.5 months and make up the majority of the ATL cases. ATL cells are often positive for FoxP3 which is an essential T regulatory marker and could explain the immunosuppression commonly found in ATL patients.

HAM/TSP is a chronic inflammatory disease of the central nervous system. Afflicted patients experience a progressive spastic weakness of the legs, lower back pain, and bowel/bladder dysfunction. Central Nervous System (CNS) damage such as spinal cord lesions and myelin loss are induced through a combination of direct viral cytopathic effects, and by immune mediated reactions. Despite the immune system targeting HTLV-1 infected cells, it is typically unable to clear the virus and the chronic inflammatory state causes progressive damage to the CNS resulting in paralysis.

The HTLV genome follows the canonical structure of replication competent retroviruses in contain gag, pol, and env domains flanked by two long terminal repeat (LTR) domains on either end of the provirus. The pX between the env and 3′ LTR encodes several alternatively spliced regulatory genes with the two most heavily implicated in viral pathogenesis being HTLV-1 TAX gene and HTLV-1 basic leucine zipper (bZIP) factor (HBZ) gene.

The HTLV-1 TAX gene is located on the pX region of the HTLV-1 viral genome. It encodes a viral gene product (Tax), which is a 40 kD protein that not only mainly localizes in the nucleus, but also can be found in the cytoplasm of infected cells. TAX interacts with a variety of host proteins and is essential in transactivating the proviral transcription from the 5′ long terminal repeat (LTR). It functionally inactivates p53 and targets pRB for degradation. It dysregulates several pathways including; NF-kB, cyclic AMP response element-binding protein (CREB), serum responsive factor (SRF) and activator protein 1 (AP-1). The pleiotropic functions of TAX all contribute to the viral pathogenicity and transformation of infected cells.

HBZ is also a nuclear protein but can be found in the cytoplasm and has 3 domains; an activation domain, a central domain, and a basic leucine zipper domain. HBZ is an antagonist to many TAX-mediated function and is essential for viral persistence and immune evasion as overexpressed TAX is a target for the CTLs. In the activation domain of HBZ there are two LXXLL-like motifs that bind to the KIX domain of CBP/p300, important transcription coactivators. These motifs are also required for HBZ to activate TGF-b/Smad signaling which is critical for HBZ induced Foxp3 expression.

The lack of effective treatment options for HTLV-1 associated diseases is an unfortunate situation and a cure or effective vaccine is in dire need for affected communities. The interest of developing an HTLV-1 vaccine began in the 1980s. A vaccinia vector expressing the HTLV-1 envelope gene could induce partial protection against HTLV-1 infection in rodents and passive immunity can be granted with an anti-HTLV-1 gp46 antibody. Therapeutic vaccines using TAX antigens induced sustained immunes responses in ATL patients stabilizing disease progressions or even inducing partial remission and HBZ vaccines were shown to elicit T cell responses and clear HBZ induced lymphoma in mouse models. As such, there remains a need for an effective vaccine for HTLV-1.

### SUMMARY OF THE INVENTION

It is against the above background that the present invention provides certain advantages over the prior art.

Although this invention as disclosed herein is not limited to specific advantages or functionalities, the invention provides a vesicular stomatitis virus (VSV)-based vaccine expressing several HTLV-1 antigens on a single VSV vector. This recombinant VSV-HTLV-1 vaccine named VSV-gp62G-HBZΔ1-TAXΔ2 encodes HTLV1 gp62 envelope glycoprotein fused to the cytoplasmic tail of VSV-G and fused to a HBZ-TAX fusion protein encoding mutant versions of both HBZ and TAX, and does not inhibit innate immunity. This single vector encodes a unique chimeric protein and a unique fusion protein resulting in both the generation of neutralizing antibodies against HTLV-1 gp62, HBZ, and TAX, and the generation of a CTL response against HTLV-1 gp62, HBZ, and TAX.

In one aspect, the invention provides vesicular stomatitis virus (VSV) vector, wherein a gene encoding a VSV glycoprotein G (VSV G) is substituted with an engineered gene encoding a chimeric glycoprotein, wherein the chimeric glycoprotein comprises an amino-terminal amino acid sequence from human T-cell leukemia virus type 1 (HTLV-1) gp62 protein and a carboxy-terminal amino acid sequence from the VSV G.

In one aspect of the VSV vector, the chimeric glycoprotein comprises at least 70% sequence identity to the amino acid sequence set forth in SEQ ID NO:30.

In one aspect of the VSV vector, the vector further comprises an engineered gene encoding a fusion protein of HTLV-1 basic leucine zipper (bZIP) factor (HBZ) and HTLV-1 TAX.

In one aspect of the VSV vector, the fusion protein comprises at least 70-99% sequence identity to the amino acid sequence set forth in SEQ ID NO:2.

In one aspect of the VSV vector, the fusion protein comprises at least 95% sequence identity to the amino acid sequence set forth in SEQ ID NO:18.

In one aspect of the VSV vector, the fusion protein comprises at least 70-99% sequence identity to the amino acid sequence set forth in SEQ ID NO:6.

In one aspect of the VSV vector, the fusion protein comprises at least 95% sequence identity to the amino acid sequence set forth in SEQ ID NO:20.

In one aspect of the VSV vector, HTLV-1 HBZ is at an amino-terminus of the fusion protein and HTLV-1 TAX is at a carboxy-terminus of the fusion protein.

In one aspect of the VSV vector, the fusion protein comprises at least 70% sequence identity to the amino acid sequence set forth in SEQ ID NO:26.

In one aspect of the VSV vector, the fusion protein comprises at least 95% sequence identity to the amino acid sequence set forth in SEQ ID NO:28.

In one aspect of the VSV vector, the fusion protein is encoded in the G-L transgene site of the VSV vector.

In another aspect, the invention provides a vaccine, comprising the VSV vector as disclosed herein.

In an aspect of the vaccine, the vaccine is administered with an adjuvant.

The invention also provides a method of producing an immune response against HTLV-1, comprising administering to a subject in need thereof the VSV vector or the vaccine as disclosed herein.

In one aspect of the method, the VSV vector or the vaccine is administered, for example, by intramuscular (IM) injection, subcutaneous (SC) injection, intradermal (ID) injection, oral administration, mucosal administration, or intranasal application.

In one aspect of the method, the subject is infected with HTLV-1.

In one aspect of the method, the subject was exposed to HTLV-1.

In one aspect of the method, the subject is not infected with HTLV-1.

In one aspect of the method, the immune response comprises the subject generating antibodies to HTLV-1 gp62, HTLV-1 TAX, and/or HTLV-1 HBZ.

In one aspect of the method, the immune response comprises the subject generating cytotoxic T cells (CTL) to HTLV-1 gp62, HTLV-1 TAX, and/or HTLV-1 HBZ.

The invention also provides a host cell comprising the VSV vector as disclosed herein.

The invention further provides a fusion protein comprising HTLV-1 TAX and HTLV-1 basic leucine zipper (bZIP) factor (HBZ).

In one aspect of the fusion protein, the fusion protein comprises at least 70-99% sequence identity to the amino acid sequence set forth in SEQ ID NO:2.

In one aspect of the fusion protein, the fusion protein comprises at least 95% sequence identity to the amino acid sequence set forth in SEQ ID NO:18.

In one aspect of the fusion protein, the fusion protein comprises at least 70-99% sequence identity to the amino acid sequence set forth in SEQ ID NO:6.

In one aspect of the fusion protein, the fusion protein comprises at least 95% sequence identity to the amino acid sequence set forth in SEQ ID NO:20.

In one aspect of the fusion protein, HTLV-1 HBZ is at the amino terminus of the fusion protein and HTLV-1 TAX is at the carboxy terminus of the fusion protein.

In one aspect of the fusion protein, the fusion protein comprises at least 70% sequence identity to the amino acid sequence set forth in SEQ ID NO:26.

In one aspect of the fusion protein, the fusion protein comprises at least 95% sequence identity to the amino acid sequence set forth in SEQ ID NO:28.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1A-1D: Designing the TAX and HBZ mutant proteins. (FIG. 1A) Sequence alignment of TAX (SEQ ID NO:2), TAX-NF-kB (SEQ ID NO:3) and TAX CREB-ATF (SEQ ID NO:4). (FIG. 1B) 293T cells were cotransfected with a constitutively active RIG (ΔRIG), the indicated Firefly luciferase reporter plasmid, TK renilla luciferase, and either empty vector (EV) of pCDNA 3.1, HTLV-1 TAX, TAX-NF-kB and TAX CREB-ATF, luciferase activity was analyze 24 hours post transfection. (FIG. 10) Sequence alignment of HBZ and 5 novel HBZ mutants designated with their respective tandem Alanine mutations (HBZ is SEQ ID NO:6; HBZΔ27 is SEQ ID NO:8; HBZA124 is SEQ ID NO:10; HBZΔ73 is SEQ ID NO:12; HBZΔ180 is SEQ ID NO:14; HBZΔ115 is SEQ ID NO:16). (FIG. 1D) 293T cells were cotransfected with a constitutively active RIG-I (ΔRIGI), the indicated Firefly luciferase reporter plasmid, TK renilla luciferase, and either EV, HTLV-1 HBZ, or the designated mutant HBZ.

FIG. 2A-2F: Construction and expression of HTLV-1 TAX and HBZ fusion mutants. (FIG. 2A) Diagram showing the chosen mutations of HTLV-1 proteins TAX and HBZ with the tandem alanine mutations. (FIG. 2B) Immunoblot analysis of transfected 293T cells with either wild type HTLV proteins TAX and HBZ alongside the mutant versions. (FIG. 2C) Diagram showing the 4 novel TAX-HBZ fusion proteins with their respective orientation of either wildtype or mutant versions. (FIG. 2D) Immunoblot analysis of 293T transfected with the fusion TAX-HBZ proteins and relative expression levels of each. (FIG. 2E) 293T cells were cotransfected with ΔRIGI and 100 ng of the indicated HTLV-1 proteins with either IFNβ. (left) or NF-kB (right) reporter. (FIG. 2F) Wildtype MEFs and (FIG. 2E) hTERT-BJ1 cells were infected with VSV-XN2, VSVm (DTY-_{AAA52-54}), or VSVm-HBZΔ1-TAXΔ2 at the indicated MOI and IFNβ levels were measured by ELISA 24 hours post infection (hpi).

FIG. 3A-3E: Creation of rVSV-HTLV-1 vaccines and expression. (FIG. 3A) Diagram depicting the arrangement of the gp62G glycoprotein with a model of its placement within the VSV virion and its corresponding vector map showing the genome arrangement of the VSV vectors used. (FIG. 3B) TEM images of cell-free VSV-XN2 and VSV-gp62G-HBZΔ1-TAXA2 and the dimensions of each. (FIG. 3C) Micrographs of HEK293 cells infected with VSV-XN2 and VSV-gp62G-HBZΔ1-TAXA2 at MOI 15 hpi show distinct CPE in response to infection. (FIG. 3D) Immunoblot of HEK293T cells infected with VSVXN2 and VSVgp62G HBZΔ1-TAXA2 at MOI 1 and harvested 5 hpi (10 μg/lane). (FIG. 3E) Growth kinetic assay of HEK293 cells infected with VSV-XN2 or VSV-gp62G-HBZΔ1-TAXA2× at MOI 0.001 and supernatant was collected 2, 16, 24, 40, and 48 hpi and viral titer was determined using Vero cells.

FIG. 4A-4C: VSV-gp62G-HBZΔ1-TAXA2 is capable of infecting primary murine Murine Embryonic Fibroblasts (MEFs). (FIG. 4A) Micrographs of MEFs isolated from wildtype C57/BL6 cells and infected with VSV-XN2 or VSV-gp62G-HBZΔ1-TAXA2 at MOI 5 and taken at 24 hpi. (FIG. 4B) Immunoblot of MEF cells infected with either VSV-XN2 or VSV-gp62G-HBZΔ1-TAXA2 at MOI 5 and harvested 24 hpi. (FIG. 4C) Growth kinetic assay from MEF cells infected with MOI 0.05 of either VSV-XN2 or VSV-gp62G-HBZΔ1-TAXA2 and supernatant was collected 2, 24, 48 hpi. Viral titer was analyzed by plaque assay with Vero cells.

FIG. 5A-5D: VSV-gp62G-HBZΔ1-TAXΔ2 is capable inducing neutralizing antibodies against HTLV-1 env and antibodies against HTLV-1 TAX. (FIG. 5A) C57 mice were vaccinated in Prime-Boost strategy on Day 0 and Day 23. On Day 7 and 30 a portion of vaccinated mice were sacrificed and serum and splenocytes were collected. (FIG. 5B) Indirect ELISA was used to detect antibodies in serum of mice for HTLV-1 env (left) or HTLV-1 TAX (right). (FIG. 5C) Syncytia neutralization assay was performed to determine if gp62 antibodies could prevent syncytia formation between MT2 cells and K562 cells transfected with VCAM1 at different dilutions (1:10 shown in 5C). (FIG. 5D) Quantitation of syncytia observed in (FIG. 5C) Syncytia was counted if diameter was more than twice that of a normal cell.

FIG. 6A-6D: Cytotoxic T cell analysis from vaccinated mice. (FIG. 6A) CD8 T cells were isolated from the spleens of vaccinated mice (7 days post prime, 6A-6C or 7 days post boost 6D, 6E) from the previous figure. CD8 T cells from each group were incubated with overlapping peptides of HTLV-1 TAX, HBZ, or gp62 env at 10 μg/ml (left) or with HBZ peptide pool at 20 μg/ml (right) and IFNγ secreting cells were determined using ELISPOT. (FIG. 6B) Splenocytes isolated from vaccinated mice were incubated 3 days with peptides at 10 μg/ml and then stained for CD8 and IFNγ using Brefelding A and analyzed by flow cytometry. (FIG. 6C) CD8 T cells from vaccinated mice (7 days post boost) were treated as in FIG. 6A. (FIG. 6D) Splenocytes from boosted mice (7 days post boost) were treated as in FIG. 6B.

FIG. 7A-7C: VSV-GFP, VSV-gp62G-GFP and VSV-gp62G-HBZΔ1-TAXΔ2 are capable of infecting and inducing death in ATL cells compare to primary lymphocytes. (FIGS. 7A, 7B and 7C) ATL cells were infected with either VSV-GFP or VSV-gp62G-GFP and VSV-gp62G-HBZΔ1-TAXΔ2 at an MOI of 1 or 0.1. (FIG. 7A) Fluorescent microscopy of infected ATL cells and primary lymphocytes was realized at 20 hpi with VSV-GFP and 50 hpi with VSV-gp62G-GFP. (FIGS. 7B and 7C) Cells were collected at 20 hpi. The percentage of infected cells (GFP+) with VSV-GFP and VSV-gp62G-GFP (FIG. 7B) was measured and cell death was determined using fixable viability dye with VSV-GFP, VSV-gp62G-GFP and VSV-gp62G-HBZΔ1-TAXΔ2 (FIG. 7C) by flow cytometry.

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Before describing the present invention in detail, a number of terms will be defined. As used herein, the singular forms “a,” “an,” and “the” include plural referents unless the context clearly dictates otherwise. For example, reference to a “nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

As used herein, the terms “polynucleotide,” “nucleotide,” “oligonucleotide,” and “nucleic acid” can be used interchangeably to refer to nucleic acid comprising DNA, RNA, derivatives thereof, or combinations thereof, in either single-stranded or double-stranded embodiments depending on context as understood by the skilled worker.

As used herein, the terms “recombinant gene” or “engineered gene” refer to a gene or DNA sequence that is augmented by the hand of man. Thus, a recombinant gene can be a DNA sequence from another species or can be a DNA sequence that originated from or is present in the same species but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA. In some aspects, said recombinant genes are encoded by cDNA. In other embodiments, recombinant genes are synthetic and/or codon-optimized for expression in a host organism.

In some embodiments, this disclosure relates to vesicular stomatitis virus (VSV) vectors, however, other vectors may be contemplated in other embodiments including, but not limited to, prime boost administration comprising administration of a recombinant VSV vector in combination with another recombinant vector expressing one or more HTLV-1 proteins, antigens, genes, or epitopes. Examples of alternative viral vector-based vaccines can include, but is not limited to, retroviruses, lentiviruses, adenoviruses, adeno-associated viruses, cytomegalovirus vectors, sendai virus vectors, alphaviruses, poxviruses, vaccinia viruses, or combinations thereof.

VSV is a practical, safe, and immunogenic vector, and an attractive candidate for developing vaccines for use in humans. VSV is a member of the Rhabdoviridae family of enveloped viruses containing a non-segmented, negative-sense RNA genome. The genome is composed of 5 genes arranged sequentially 3′-N-P-M-G-L-5′, each encoding a polypeptide found in mature virions. Notably, the surface glycoprotein G is a transmembrane polypeptide that is present in the viral envelope as a homotrimer, and like Env, it mediates cell attachment and infection. In certain embodiments, the VSV G is replaced by the HTLV-1 glycoprotein gp62. In some embodiments, the VSV G is partially replaced by the HTLV-1 glycoprotein gp62 to make a chimeric glycoprotein comprising the amino portion of the HTLV-1 gp62 and the carboxy portion of the VSV G.

In one embodiment, the invention provides a vesicular stomatitis virus (VSV) vector, wherein a gene encoding a VSV glycoprotein G (VSV G) is substituted with an engineered gene encoding a chimeric glycoprotein, wherein the chimeric glycoprotein comprises an amino-terminus of human T-cell leukemia virus type 1 (HTLV-1) gp62 protein and a carboxy-terminus of the VSV G. In one aspect of this embodiment, the chimeric glycoprotein comprises a chimeric glycoprotein having at least 70% sequence identity to the amino acid sequence set forth in SEQ ID NO:30. In another aspect, the vector further comprises an engineered gene encoding a fusion protein of HTLV-1 basic leucine zipper (bZIP) factor (HBZ) and HTLV-1 TAX. In yet another aspect, the fusion protein comprises a TAX mutant protein having at least 70-99% sequence identity to the amino acid sequence set forth in SEQ ID NO:2. In yet another aspect of the fusion protein, the fusion protein comprises a TAX mutant protein having at least 95% sequence identity to the amino acid sequence set forth in SEQ ID NO:18. In yet another aspect, the fusion protein comprises a HBZ mutant protein having at least 70-99% sequence identity to the amino acid sequence set forth in SEQ ID NO:6. In yet another aspect of the fusion protein, the fusion protein comprises a HBZ mutant protein having at least 95% sequence identity to the amino acid sequence set forth in SEQ ID NO:20. In yet another aspect, HTLV-1 HBZ is at an amino terminus of the fusion protein and HTLV-1 TAX is at a carboxy terminus of the fusion protein. In yet another aspect, the fusion protein comprises a fusion protein having at least 70% sequence identity to the amino acid sequence set forth in SEQ ID NO:26. In yet another aspect of the fusion protein, the fusion protein comprises a fusion protein having at least 95% sequence identity to the amino acid sequence set forth in SEQ ID NO:28. In yet another aspect, the fusion protein is encoded in the G-L transgene site of the VSV vector.

In another embodiment, the invention provides a vesicular stomatitis virus (VSV) vector, wherein a gene encoding a VSV glycoprotein G (VSV G) is substituted with an engineered gene encoding a chimeric glycoprotein, wherein the chimeric glycoprotein comprises an amino-terminus of human T-cell leukemia virus type 1 (HTLV-1) gp62 protein and a carboxy-terminus of the VSV G. In one aspect of this embodiment, the chimeric glycoprotein comprises a chimeric glycoprotein having at least 70% sequence identity to the amino acid sequence set forth in SEQ ID NO:30. In another aspect, the vector further comprises an engineered gene encoding a fusion protein of HTLV-1 TAX and HTLV-1 basic leucine zipper (bZIP) factor (HBZ). In yet another aspect, the fusion protein comprises a TAX mutant protein having at least 70-99% sequence identity to the amino acid sequence set forth in SEQ ID NO:2. In yet another aspect of the fusion protein, the fusion protein comprises a TAX mutant protein having at least 95% sequence identity to the amino acid sequence set forth in SEQ ID NO:18. In yet another aspect, the fusion protein comprises a HBZ mutant protein having at least 70-99% sequence identity to the amino acid sequence set forth in SEQ ID NO:6. In yet another aspect of the fusion protein, the fusion protein comprises a HBZ mutant protein having at least 95% sequence identity to the amino acid sequence set forth in SEQ ID NO:20. In yet another aspect, HTLV-1 TAX is at an amino-terminus of the fusion protein and HTLV-1 HBZ is at a carboxy-terminus of the fusion protein. In yet another aspect, the fusion protein comprises a fusion protein having at least 70% sequence identity to the amino acid sequence set forth in SEQ ID NO:24. In yet another aspect, the fusion protein is encoded in the G-L transgene site of the VSV vector.

As used herein, the term “vector” refers to a vehicle that can facilitate the transfer of nucleic acid molecules from one environment to another or that allow or facilitate the manipulation of a nucleic acid molecules. Vectors are widely used and understood by those of skill in the art, and as used herein the term “vector” is used consistent with its meaning to those of skill in the art. Any vector that allows expression of encoded HTLV-1 proteins, chimeric proteins, fusion proteins, epitopes, or antigens related to any aspect of this disclosure may be used in accordance with the present invention. In certain embodiments, the encoded proteins, chimeric proteins, fusion proteins, epitopes, or antigens of the present invention may be used in vitro (such as using cell-free expression systems) and/or in cultured cells grown in vitro in order to produce the encoded HTLV-1 proteins, chimeric proteins, fusion proteins, epitopes, or antigens which may then be used for various applications such as in the production of proteinaceous vaccines. For such applications, any vector that allows expression of the HTLV-1 encoded proteins, chimeric proteins, fusion proteins, epitopes, or antigens in vitro and/or in cultured cells may be used.

As used herein, the terms “vector” or “recombinant expression construct” can be used interchangeably, and refer to a construct or an engineered construct that allows expression of the HTLV-1 encoded proteins, chimeric proteins, fusion proteins, epitopes, or antigens in cultured cells.

As used herein, the term “immune response” or “immunogenic” refers to the ability of HTLV-1 encoded proteins, chimeric proteins, fusion proteins, epitopes, or antigens to stimulate or elicit an immune response in a subject. An immune response can be measured, for example, by determining the presence of antibodies specific for the HTLV-1 encoded proteins, chimeric proteins, fusion proteins, epitopes, or antigens. The presence of antibodies can be detected by methods known in the art, for example using an ELISA assay. An immune response can also be measured, for example, by determining the presence of cytotoxic T lymphocytes (CTL) specific for the HTLV-1 encoded proteins, chimeric proteins, fusion proteins, epitopes, or antigens. The presence of CTLs can be detected by methods known in the art, for example using splenocytes isolated from a vaccinated subject and performing an ELISPOT assay. Cytotoxic T lymphocytes are generated by immune activation of cytotoxic T cells (Tc cells), and CTLs are generally CD8+. CTLs are able to eliminate most cells in the body since most nucleated cells express class I MHC molecules.

As used herein, the terms “chimeric protein” or “chimeric polypeptide” can be used interchangeably, and refer to an engineered glycoprotein formed through the combination of portions of at least two or more coding sequences to produce a new gene that encodes the amino acid sequences from the at least two different glycoproteins. In certain embodiments, the amino acid sequences from the at least two different glycoproteins can include regions or domains of each glycoprotein, for example, an extracellular domain, a transmembrane domain, one or more stimulatory domains, and/or an intracellular domain. The glycoproteins of the present invention can be prepared by expression in an expression vector as a chimeric protein. The methods to produce a chimeric protein comprising an HTLV-1 glycoprotein and a VSV G glycoprotein are known to those with skill in the art. In some embodiments, the chimeric glycoprotein comprises a portion of the HTLV-1 gp62 and a portion of the VSV G. In certain embodiments, a chimeric glycoprotein comprises the amino portion of the HTLV-1 gp62 and the carboxy portion of the VSV G.

In one embodiment, a chimeric glycoprotein comprises the amino portion (i.e., residues 1-463) of the HTLV-1 gp62 and the carboxy portion (i.e., the final at least 12 residues, or the last 23 residues) of the VSV G.

The invention further provides a fusion protein comprising HTLV-1 basic leucine zipper (bZIP) factor (HBZ)HTLV-1 TAX. In one aspect of the fusion protein, the fusion protein comprises a TAX mutant protein having at least 70-99% sequence identity to the amino acid sequence set forth in SEQ ID NO:2. In yet another aspect of the fusion protein, the fusion protein comprises a TAX mutant protein having at least 95% sequence identity to the amino acid sequence set forth in SEQ ID NO:18. In one aspect of the fusion protein, the fusion protein comprises a HBZ mutant protein having at least 70-99% sequence identity to the amino acid sequence set forth in SEQ ID NO:6. In yet another aspect of the fusion protein, the fusion protein comprises a HBZ mutant protein having at least 95% sequence identity to the amino acid sequence set forth in SEQ ID NO:20. In one aspect of the fusion protein, HTLV-1 HBZ is at the amino terminus of the fusion protein and HTLV-1 TAX is at the carboxy terminus of the fusion protein. In one aspect of the fusion protein, the fusion protein comprises a fusion protein having at least 70% sequence identity to the amino acid sequence set forth in SEQ ID NO:26. In yet another aspect of the fusion protein, the fusion protein comprises a fusion protein having at least 95% sequence identity to the amino acid sequence set forth in SEQ ID NO:28.

As used herein, the term “fusion protein” refers to a fusion of two or more gene sequences into an engineered, non-natural single reading frame to encode the fusion protein as a single transcript (i.e., encoding a single polypeptide comprising two functional segments). The individual proteins merged into a fusion protein often retain their original functions. In some embodiments, the fusion proteins as disclosed herein can comprise a HTLV-1 HBZ sequence linked, fused, or conjugated to a HTLV-1 TAX sequence. In certain embodiments, the fusion protein as disclosed herein is comprised of HTLV-1 HBZ and HTLZ1 TAX mutants or variants thereof.

As used herein, the terms “variant” and “mutant” are used interchangeably herein except that a “variant” is typically non-recombinant in nature, whereas a “mutant” is typically recombinant. For example, a variant or a mutant HTLV-1 HBZ or a variant or a mutant HTLV-1 TAX can encompass polypeptides having at least 70%, at least 75%, at least 78%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to a wild type HTLV-1 HBZ sequence or a wild type HTLV-1 TAX sequence or corresponding fragment thereof. In some embodiments, a mutant HTLV-1 HBZ or a mutant HTLV-1 TAX can be mutated at one or more amino acids in order to modulate its therapeutic or immunogenic efficacy. In certain embodiments, a mutant contains a substitution, deletion and/or insertion at an amino that is known to modulate its therapeutic or immunogenic efficacy. In other embodiments, a mutant contains a substitution, deletion and/or insertion at an amino that is a conserved amino acid present in a wild type HBZ or TAX protein. In certain embodiments, a mutant has no more than 75, 50, 40, 30, 25, 20, 15, 12, 10, 9, 8, 7, 6, 5, 4, 3, 2, 1 amino acid differences as compared to the reference or wild-type sequence.

As used herein, the terms “linker” or “linker domain” or “linked” refer to an oligo- or polypeptide region from about 1 to 100 amino acids in length, which links together any of the domains/regions of the fusion protein of the invention. Linkers may be composed of flexible residues like glycine and serine so that the adjacent protein domains are free to move relative to one another. For example, an exemplary Gly/Ser peptide linker comprises the amino acid sequence (Gly₄Ser)ₙ, wherein n is an integer that is the same or higher than 1, 2, 3, 4, 5, 6, 7, 8, 9, 10, 15, 20, 25, 30, 35, 40, 50, or 100. Longer linkers may be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. Other linkers will be apparent to those of skill in the art and may be used in connection with alternate embodiments of the invention. In other embodiments, the fusion protein does not contain a linker.

In another aspect, the invention provides a vaccine, comprising the VSV vector as disclosed herein. In an aspect of the vaccine, the vaccine is administered with an adjuvant. In some embodiments, the vaccine can be formulated for administration in one of many different modes. One skilled in the art will recognize that although more than one route can be used for administration, a particular route can provide a more immediate and more effective reaction than another route. Routes of administration of any of the compositions of the invention can include, but are not limited to, inhalation, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In certain embodiments, the vaccine can be administered by intramuscular (IM) injection, subcutaneous (SC) injection, intradermal (ID) injection, oral administration, mucosal administration, or intranasal application. The vaccine composition can contain a variety of additives, such as adjuvant, excipient, stabilizers, buffers, or preservatives. In some embodiments, the vaccine composition can be supplied in a vessel appropriate for distribution. Administration of the vector and/or vaccine may consist of a single dose or a plurality of doses over a period of time.

HTLV-1 is a retrovirus that is very efficient at evading the immune system. Following infection of a new host, HTLV-1 infects cells through its glycoprotein gp62. It then achieves latency through integration into the host genome and increases proviral load mainly through proliferation of infected cells. HBZ is a key regulatory protein capable of downregulating viral gene expression and helping the virus achieve persistent latency while TAX induces viral expression via LTR activation mediated through CREB/ATF. Both TAX and HBZ have been implicated in HTLV-1 pathogenesis and are possible targets in vaccine design. Previous studies have indicated that vaccination with rVV expressing HTLV-1 gp62 could achieve lasting immunity against HTLV-1 infection in cynomolgus monkeys, thus, the envelope protein is also a target of vaccine design in preventing HTLV-1 infection. The vector disclosed herein is the only vaccine design to the inventor's knowledge that encodes both the HTLV-1 envelope gp62 and nonstructural viral proteins like TAX and HBZ.

TAX and HBZ are implicated in disease progression and the strategy is a targeted mutation of key domains allowing minimal alteration of the amino acid sequence from the wildtype version while disrupting the immunosuppressive phenotype of TAX and HBZ. It was found the HBZΔ1-TAXΔ2 mutant did not inhibit IFN promoter activity when transfected into 293T cells expressing an active RIGI mutant; however, the wildtype TAX and HBZ and the wildtype fusion variant heavily inhibited IFN promoter activity. Additionally, by coupling TAX and HBZ expressing into a single polypeptide, TAX and HBZ function was significantly disrupted. TAX and HBZ function in opposing roles in the context of HTLV-1 pathogenesis. TAX mediates oncogenesis through chronic NF-kB activation, while HBZ suppresses NF-kB activity. By coupling the proteins together, it was discovered that NF-kB promoter activity is neither completely suppressed, nor highly regulated as with TAX overexpression.

The VSV-gp62G-HBZΔ1-TAXΔ2 vaccine was capable of inducing a significant humoral response to the envelope protein and the antibodies produced demonstrated significant neutralizing activity using the syncytia assays. Additionally, a TAX antibody response was observed, indicating that viral gene expression occurred in vaccinated animals. CTL analysis indicated a significant cell mediated immune response following peptide stimulation. This demonstrates that VSVgp62G-HBZΔ1-TAXΔ2 can be a valuable vaccine vector in both a prophylactic and therapeutic agent and warrants further testing in additional animal models.

The invention also provides a host cell comprising the VSV vector as disclosed herein. The vectors and/or vaccines of the invention can be delivered to cells, for example if the aim is to express the HTLV-1 proteins, chimeric proteins, fusion proteins, epitopes, or antigens in cells in order to produce and isolate the expressed proteins, such as from cells grown in culture. For expressing the HTLV-1 proteins, chimeric proteins, fusion proteins, epitopes, or antigens in cells, any suitable transfection, transformation, or gene delivery methods can be used. Such methods are well known by those skilled in the art, and one of skill in the art would readily be able to select a suitable method depending on the nature of the nucleotide sequences, vectors, and cell types used. For example, transfection, transformation, microinjection, infection, electroporation, lipofection, or liposome-mediated delivery could be used. Expression of the HTLV-1 proteins, chimeric proteins, fusion proteins, epitopes, or antigens can be carried out in any suitable type of host cells, such as bacterial cells, yeast, insect cells, and mammalian cells. The HTLV-1 proteins, chimeric proteins, fusion proteins, epitopes, or antigens of the invention can also be expressed using including in vitro transcription/translation systems. All of such methods are well known by those skilled in the art, and one of skill in the art would readily be able to select a suitable method depending on the nature of the vaccines, vectors, and cell types used.

The chimeric proteins and/or the fusion proteins as disclosed herein, can be used in any number of vaccine types including, but not limited to live-attenuated vaccines, inactivated vaccines, subunit vaccines, recombinant vector vaccines, polysaccharide vaccines, conjugate vaccines, and DNA vaccines.

In certain embodiments, the HTLV-1 proteins, chimeric proteins, fusion proteins, epitopes, or antigens of the invention are administered in vivo, for example where the aim is to produce an immunogenic response in a subject. A “subject” in the context of the present invention may be any animal. For example, in some embodiments it may be desired to express HTLV-1 proteins, chimeric proteins, fusion proteins, epitopes, or antigens of the invention in a laboratory animal, such as for pre-clinical testing of the HTLV-1 immunogenic compositions and vaccines. In other embodiments, it will be desirable to express the HTLV-1 proteins, chimeric proteins, fusion proteins, epitopes, or antigens of the invention in human subjects, such as in clinical trials and for actual clinical use of the immunogenic compositions and vaccine of the invention. In certain embodiments, the subject is a human, for example a human that is infected with, or is at risk of infection with, HTLV-1.

In certain embodiments, adjuvants may also be included. Adjuvants include, but are not limited to, mineral salts (e.g., AlK(SO₄)₂, AlNa(SO₄)₂, AlNH(SO₄)₂, silica, alum, Al(OH)₃, Ca₃(PO₄)₂, kaolin, or carbon), polynucleotides with or without immune stimulating complexes (ISCOMs) (e.g., CpG oligonucleotides; poly IC or poly AU acids, polyarginine with or without CpG), JuvaVax™, certain natural substances (e.g., wax D from Mycobacterium tuberculosis, substances found in Cornyebacterium parvum, Bordetella pertussis, or members of the genus Brucella), flagellin (Toll-like receptor 5 ligand), saponins such as QS21, QS17, and QS7, monophosphoryl lipid A, in particular, 3-de-O-acylated monophosphoryl lipid A (3D-MPL), imiquimod (also known in the art as IQM and commercially available as Aldara®), and the CCR5 inhibitor CMPD167.

The invention also provides a method of producing an immune response against HTLV-1 comprising administering to a subject in need thereof the VSV vector or the vaccine as disclosed herein. In one aspect of the method, the VSV vector or the vaccine is administered by intramuscular (IM) injection, subcutaneous (SC) injection, intradermal (ID) injection, oral administration, mucosal administration, or intranasal application. In one aspect of the method, the subject is infected with HTLV-1. In one aspect of the method, the subject was exposed to HTLV-1. In one aspect of the method, the subject is not infected with HTLV-1.

In one aspect of the method, the immune response comprises the subject generating antibodies to HTLV-1 gp62, HTLV-1 TAX, and/or HTLV-1 HBZ. In one aspect of the method, the immune response comprises the subject generating cytotoxic T cells (CTL) to HTLV-1 gp62, HTLV-1 TAX, and/or HTLV-1 HBZ.

Suitable dosages of the vaccines and/or vectors of the invention in an immunogenic composition of the invention can be readily determined by those of skill in the art. For example, the dosage of the vaccines and/or vectors can vary depending on the route of administration and the size of the subject. Suitable doses can be determined by those of skill in the art, for example by measuring the immune response of a subject, such as a laboratory animal, using conventional immunological techniques, and adjusting the dosages as appropriate. Such techniques for measuring the immune response of the subject include, but are not limited to, chromium release assays, tetramer binding assays, IFN-.gamma. ELISPOT assays, IL-2 ELISPOT assays, intracellular cytokine assays, and other immunological detection assays, e.g., as detailed in the text “Antibodies: A Laboratory Manual” by Ed Harlow and David Lane.

When provided prophylactically, the immunogenic compositions of the invention are ideally administered to a subject in advance of HTLV-1 infection, or evidence of HTLV-1 infection, or in advance of any symptom due to HTLV-1, especially in high-risk subjects. The prophylactic administration of the vectors and/or vaccines can serve to provide protective immunity of a subject against HTLV-1 infection or to prevent or attenuate the progression of HTLV-1 in a subject already infected with HTLV-1. When provided therapeutically, the immunogenic compositions can serve to ameliorate and treat HTLV-1 symptoms and are advantageously used as soon after infection as possible, preferably before appearance of any symptoms of HTLV-1 infection or progression, but may also be used at (or after) the onset of the disease symptoms.

Immunization schedules (or regimens) are well known for animals (including humans) and can be readily determined for the particular subject and immunogenic composition. Hence, the vaccines and/or vectors can be administered one or more times to the subject. In certain embodiments, there is a set time interval between separate administrations of the vaccines and/or vectors. While this interval varies for every subject, typically it ranges from 10 days to several weeks, and is often 2, 4, 6 or 8 weeks. For humans, the interval is typically from 2 to 6 weeks. The immunization regimes typically have from 1 to 6 administrations of the vaccines and/or vectors, but may have as few as one or two or four. The methods of inducing an immune response can also include administration of an adjuvant with the immunogens. In some instances, annual, biannual or other long interval (5-10 years) booster immunization can supplement the initial immunization protocol.

In certain embodiments, the methods as disclosed herein can include a variety of prime-boost regimens, for example DNA prime-Adenovirus boost regimens. In these methods, one or more priming immunizations are followed by one or more boosting immunizations. The actual immunogenic composition can be the same or different for each immunization and the type of immunogenic composition (e.g., the HTLV-1 proteins, chimeric proteins, fusion proteins, epitopes, or antigens of the invention), the route, and formulation of the immunogens can also be varied. For example, if a vector is used for the priming and boosting steps, it can either be of the same or different type (e.g., DNA or bacterial or viral expression vector). One useful prime-boost regimen provides for two priming immunizations, four weeks apart, followed by two boosting immunizations at 4 and 8 weeks after the last priming immunization. It should also be readily apparent to one of skill in the art that there are several permutations and combinations that are encompassed using the DNA, bacterial and viral expression vectors of the invention to provide priming and boosting regimens.

The invention will be further described in the following examples, which do not limit the scope of the invention described in the claims.

### Examples

The Examples that follow are illustrative of specific embodiments of the invention, and various uses thereof. They are set forth for explanatory purposes only, and are not to be taken as limiting the invention.

### Example 1: Materials and Methods

### Cells

293T cells (human embryonic kidney epithelial cells; American Type Culture Collection, Vero cells (immortalized Cercopithecus aethiops kidney epithelial cells; ATCC) and Mouse embryonic fibroblasts (MEFs) were maintained in DMEM (Life Technologies/Invitrogen) supplemented with 10% FBS and 5% penicillin-streptomycin. HEK293 cells were maintained in MEM medium (Gibco/Invitrogen) supplemented with 10% FBS and 5% penicillin-streptomycin. EL4 (mouse T lymphocyte) cells were maintained in RPMI 1640 Medium (Gibco/Invitrogen) supplemented with 10% FBS, 50 μM β-Mercapto Ethanol and 5% penicillin-streptomycin. K562 cells (human bone marrow lymphoblast were maintained in RPMI 1640 Medium (Gibco/Invitrogen) supplemented with 10% FBS, 2 mM L-Glutamine and 5% penicillin-streptomycin. hTERT BJ1 were maintained in a 4:1 ratio of DMEM: Medium 199 with 10% FBS, 1 mM sodium pyruvate, and 4 mM L-glutamine.

### Luciferase Reporter Gene Assays

For reporter gene assays, 293T cells were placed in 48-well plates and transiently transfected with 100 ng of luciferase reporter plasmid, 10 ng of pRL-TK, 100 ng of ΔRIG-I and 100 ng of expression plasmids by using Lipofectamine 2000 (Invitrogen). After 24 hours, the cells were ruptured with cell culture lysis buffer (Promega) and luciferase activity was measured using a luminometer (TD 20/20; Turner Designs). All luciferase assay results were presented as fold induction values.

### Creation of HTLV-1 Fused Antigens and Gp62g Envelope Protein and Rvsv HTLV Vaccines

Plasmid clones that contain the HTLV TAX, mutant TAX (TAXΔ2), mutant HBZ (HBZΔ1), fusion TAX-HBZ, fusion TAXΔ2-HBZΔ1, fusion HBZ-TAX, fusion HBZΔ1-TAXΔ2, and gp46G were purchased from Genscript and cloned into pCDNA 3.1. The HBZ plasmid was a kind gift from Dr. Ramos and had a His tag on the C-terminal end. The TAX and mutant TAX sequences were constructed using the complete cds for HTLV-1 TAX (GenBank AB038239.1) and the mutant HBZ sequences were constructed using the complete cds for HTLV-1 HBZ (GenBank: DQ273132.1). The TAXΔ2 has a tandem 6 alanine mutation amino acids mutation and the mutant HBZ sequence has a mutation to 6 tandem Alanine. The fused HTLV TAX-HBZ and HBZ-TAX and their respective mutants were flanked by 5′ XhoI and 3′ NheI restriction sites to facilitate cloning into the VSV G-L transgene site. The chimera gp62G glycoprotein was constructed using the complete cds for HTLV envelope gene (GenBank M37747.1) and contained the first 463 amino acids from the gp62 protein and the last 23 C terminal amino acids from VSV G (GenBank X0633.1). The gp62G gene was flank by a 5′ Mlu restriction site and the 3′ end included a PacI restriction site followed by the VSV transcription Stop Start sequence and the XhoI restriction site to replace the G glycoprotein. The generation of VSV and encoding gp62G glycoprotein was done using restriction digest with MluI-HF and XhoI (NEB) to create compatible ends to ligate into the VSV and VSV cDNA plasmids using Electroligase (NEB). The ligated product was transfected into DH10B E. coli and liquid cultures from colonies were grown at 30° C. overnight. DNA preps were confirmed by restriction digest and verified by sequencing reactions. The insertion of HBZΔ1-TAXΔ2 into the VSVm vector were performed similarly using XhoI and NheI for the restriction digest. Plasmid Midiprep Kits (Qiagen) were used for transfection to recover infectious virions.

### Plasmid Transfections

All plasmid transfection were done using Lipofectamine 2000 and Lipofectamine LTX (Invitrogen) following the manufacturer's recommended protocol.

Recovery and Purification of rVSV Expressing HTLV-1 Antigens

The recovery of infectious VSV virions expressing HTLV-1 proteins was performed using establish protocol. In brief, 293 Ts were plated 1.5×10⁶ cells in 6 well plates to near confluency in duplicate for each VSV construct being recovered. The next day they were infected with VVTF7-3 (vaccinia virus expressing T7 polymerase) at MOI of 1 in SF-DMEM. After 1 hour, the vaccinia was removed and DMEM containing 5% low IgG FBS was added. The cells were then transfected with VSV support plasmids and full length genome with VSV N:P:L:G being supplied with 1:1.66:0.33:2.64 μg per well and full length VSV 5 μg per well. The NPL and VSV genome plasmids are on pBSSK+ and VSV-G is on pcDNA 3.1. Transfections were performed using Lipofectamine 2000 according to the manufacturer's protocol and using a 1:1 Lipofectamine (μl): DNA (μg) ratio. The transfection mix was added to the fresh media and allowed to incubate overnight. The next day a 10 cm dish of 293T cells at roughly 50% confluency (4e6 cells were seeded and allowed to adhere overnight) was transfected with 16 μg of VSV-G using Lipofectamine 2000. The media from the VSV recovery plate was collected and passed through a 0.22 micron syringe filter twice to remove vaccinia virus. The filtered media was then passaged onto the G-complemented 293T cells. Once cytopathic effect was observed (usually 24-36 hours). The media was collected and VSV was isolated using a standard plaque isolation assay in a 6 well plate using Vero cells. Plaques were collected and virus was amplified on a 6 well plate of 293T cells. After 24 hours, if the virus was properly recovered and the cells were fully infected, the cells and media were collected and the cells were pelleted and analyzed by western blot for HTLV-1 envelope gp62, HTLV-1 TAX, HTLV-1 HBZ, and VSV-G. The media was filtered and passaged onto a 10 cm dish of 293T cells. After 24 hours, the media was collected and filter through a 0.45 μM filter and stored at −80° C. for later use.

### VSV-gp62G Ultracentrifugation

VSV-gp62G-HBZΔ1-TAXΔ2 was amplified using HEK293 cells plated to approximately 80% confluency in 15 cm dishes. The cells were inoculated with VSV gp62-HBZΔ1-TAXΔ2 at approximately 0.01 MOI diluted in serum free MEM. The cells were incubated for 1 hour and then media was removed and 15 ml of culture media. After 16-24 hours the cells were fully infected and cell media was collected, clarified through low speed centrifugation to remove cell debris and filtered through a 0.45 μm PES membrane vacuum filter. The virus was concentrated using ultracentrifugation at 27,000 RPM for 90 minutes at 4° C. and then resuspended and aliquoted and stored at −80° C.

### Virus Infections

Virus infection were done in cells were seeded in multiwell plates. Adherent cells were allowed to adhere overnight and were 80-90% confluent, unless otherwise indicated. Adherent cells were infected with rVSVs at the indicated MOI in a reduced volume of serum-free DMEM for 1 hour, with agitation at 15 minute intervals. Subsequently, cells were washed with PBS twice, and complete medium was added back to the cells. Suspension cells were collected and resuspended in serum free media. Cells were counted and transferred to a conical tube and pelleted again. Cells were resuspended in 250 μl of VSV inoculum in serum free media at the indicated MOI and incubated at 37° C. for 1 hour with tube agitation at 15 minute intervals. After 1 hour, cells were washed with PBS twice and resuspended in culture media and transferred to a culture plate.

### Immunoblots

Infected cells were collected and incubated in RIPA lysis buffer with protease inhibitor mixture (Sigma) for 30 minutes at 4° C. with gentle agitation. Cell debris was removed by centrifugation for 10 minutes at 15,000 rpm. Protein concentration was quantitated using BCA assay (Thermo Scientific), and the OD was read at 540 nm. Equal amounts of protein were separated using SDS-10% PAGE and transferred to a polyvinylidene difluoride membrane. Membranes were blocked with 5% milk powder in PBS-0.1% Tween 20 at room temperature for 1 hour and then probed with primary Abs against VSV glycoprotein (Sigma), gp46 (1c11 mouse anti-gp46 Santa Cruz), b-actin (Sigma), and HBZ (purified antibody from hybdridoma clone 3FIG1, provided by Dr. Ramos) and TAX (Santa Cruz 1A3). Membranes were then washed with PBS-0.1% Tween 20 and probed with secondary Abs. Images were resolved using an ECL system (Thermo Scientific) and detected using X-ray film

### ELISA for Mouse and Human IFNβ

Supernatant was collected from MEFs or hTERTs seeded in 24 well plates 24 hours following VSV infection from the indicated MOI. IFNβ. production was analyzed using mouse or human IFNβ. ELISA kits from PBL assay science.

### Transmission Electron Microscopy

Virus was fixed for 48 hours in 4% paraformaldehyde and kept at 4° C. until sample was loaded onto a Formvar-coated carbon copper grid and negatively stained with an aqueous solution of 1% uranyl acetate. Grids were allowed to dry overnight then viewed at 80 kV in a JEOL JEM-1400 transmission electron microscope. Images were captured by a Gatan Orius SC200 digital camera

### Growth Kinetic Assays

HEK293 cells were seeded at 1×10⁶ cells/well in 6 well dishes and infected with rVSV at the indicated MOI in serum free DMEM for 1 hour with agitation at 15 minute intervals, at the end of the incubation, the virus was removed and replaced with complete MEM.

### Mouse Studies

Female C57BL/6 mice were purchased from the Jackson Laboratory. All mice were 6-8 weeks old. Mice care and study were conducted under the approval of the Institutional Animal Care and Use Committee of the University of Miami.

### Vaccine Studies

To determine the efficacy of the rVSV-HTLV vaccine, 8-weeks old female C57BL/6 mice were vaccinated i.v. with 2×10⁶ PFU VSV-XN2 or VSV-gp62G-HBZΔ1-TAXΔ2, (n=10 per group) and boosted on day 22. Mice were bled periodically using a submandibular bleed method under isoflurane anesthesia or collected at time of sacrifice via cardiac puncture. Serum was isolated from whole blood, and Ab titer was analyzed using indirect ELISA with recombinant protein from Mybiosource.

### HTLV-1 Serum Antibody Indirect ELISA Analysis

Ninety-six—well polysterene microtiter plates were coated with recombinant HTLV-1 gp62 (2 μg/ml MyBioSource cat #MBS485161), TAX (50 ng/ml MyBioSource cat #MBS1104033) in 50 μl Bicarbonate buffer 100 mM pH 9.6 overnight at 4° C. After washing with PBS, plates were blocked with 5% BSA for 1 hour, incubated with appropriately diluted serum drawn from vaccinated or control mice for 2 hour, and incubated with HRP-conjugated anti-mouse IgG (1:5000) for 1 hour. The HRP signal was developed with TMB for 30 minutes at room temperature, and the reaction was stopped with 1 M HCl. OD was read at 450 nm on a plate reader.

### MT2-K562 Fusion Inhibition Assay

To test the serum's ability to inhibit syncytia formation, a modified syncytia inhibition assay was used. In brief, 1×10⁷ K562 cells were transfected with 18 μg of pCMV3-VCAM1-Myc using Lipofectamine LTX with PLUS Reagent and incubated overnight. The next day serum from vaccinate animals was diluted with RPMI culture media into a flat bottom 96-well and 50 μl/well was added to each well. MT2 cells were resuspended to 2×10⁶ cells/ml. The MT2 cells were aliquoted to 50 μl/well in the serum containing wells. The cells were incubated 30 minutes at room temperature and while the transfected K562 cells were suspended to 1×10⁶ and after the 30 minute incubation 100 μl of the K562 cells were added to the MT2 cells in serum media. The control wells were given culture media to a final volume of 200 μl. The cells were incubated overnight and then cell clumps were disrupted by gentle pipetting and allowed to settle for 30 minutes. Syncytia was counted using a Nikon phase contrast microscope through a 20× objective, through several different serum dilutions.

### Analysis of CTL Response in CD8 T Cells

The HTLV-specific CTL response was assessed using splenocytes isolated from vaccinated mice. CD8 T cells were isolated from whole splenocytes using MACS CD8a+ T cell isolation kit through negative selection. CD8 T Cells were plated at 2×10⁵ per well and stimulated with 20 μg/ml of overlapping 15-aa peptides covering the envelope, TAX or HBZ region of HTLV-1 (custom synthesized by GenScript). After a 72 hours stimulation, the IFNγ secreting cells were determined using an ELISPOT assay for mouse IFNγ and quantitated using the ELISPOT reader system. For flow cytometry, cells were stimulated for 72 hours. Brefeldin A (3 mg/ml) was added to the cells 6 hours before analysis. Cells were then washed, stained with cell surface marker, permeabilized with Cytofix/Cytoperm (BD Biosciences), and stained with IFNγ. Data were acquired using an LSR II flow cytometer.

### Statistical Analysis

All statistical analyses were performed using the Student t test, unless specified. The data were considered to be significantly different at p<0.05.

### Example 2: Design and Engineering of HTLV-1 Fusion Proteins

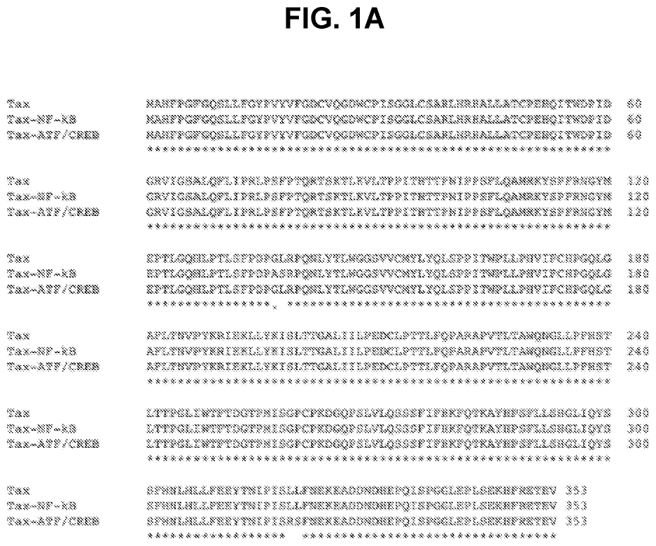

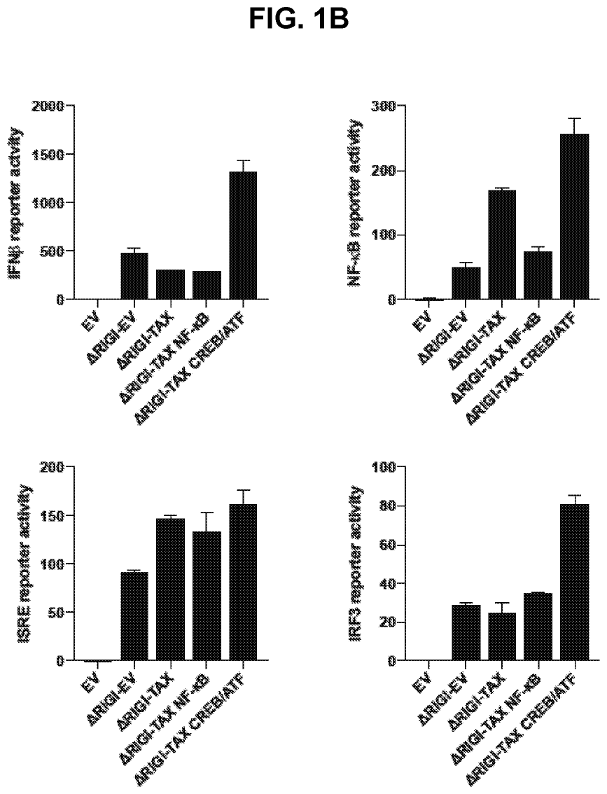

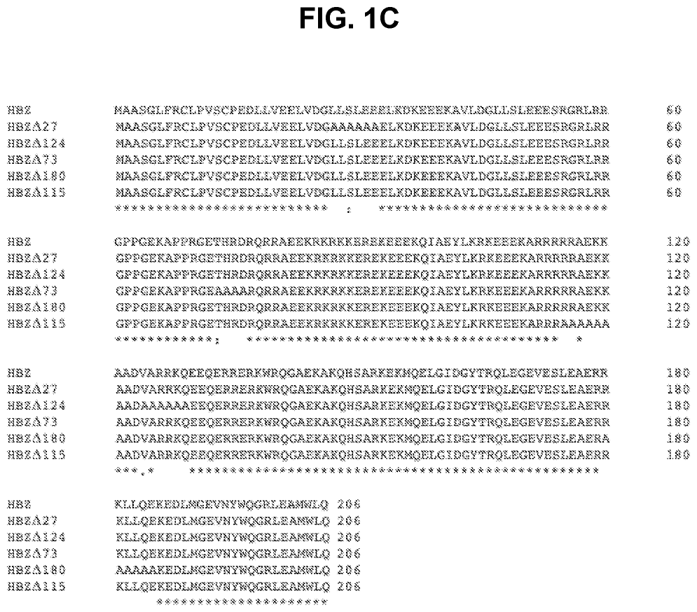

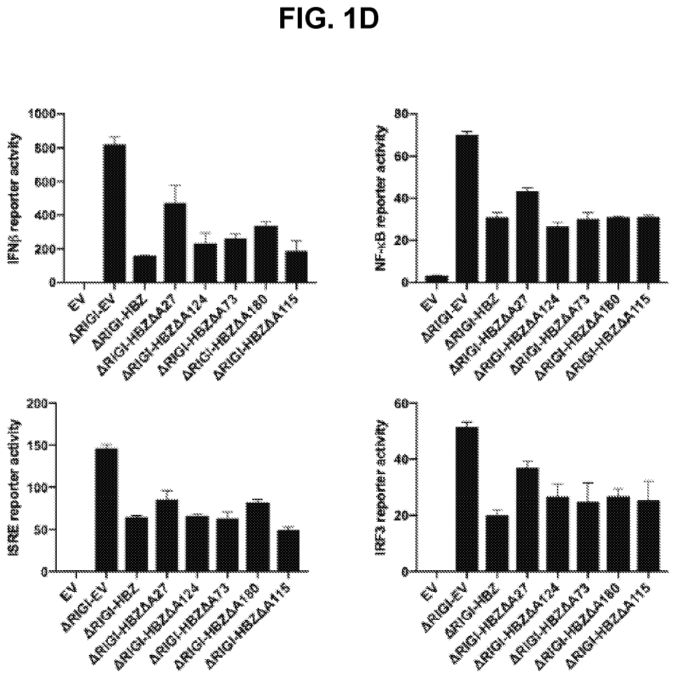

Two HTLV-1 TAX mutants were characterized, TAX NF-kB and TAX CREB/ATF. The TAX-NF-kB mutant cannot activate the NF-kB pathway and the CREB/ATF mutant lacks activity on the CREB/ATF responsive HTLV-1 LTR. Previous studies have implicated TAX as a suppressor of innate immune pathways (Hyun 2015). Here, 293T cells were cotransfected with an active RIG-I mutant (ΔRIGI) and luciferase reporters for IFNβ, NF-kB, ISRE, IRF3 alongside TAX and the two mutants. The TAX CREB/ATF mutant did not inhibit the reporter activity for these key immune pathways (FIGS. 1A and 1B). This approach was repeated using several different domains of HBZ and several mutants of HBZ were designed with tandem Alanine repeats in key domains (HBZAΔ27, HBZΔ124, HBZΔ73, HBZΔ180, HBZΔ115 (FIG. 10). The results show that the mutant HBZAΔ27 was successful in suppressing IFNβ, NF-kB, ISRE, IRF3 promoter activity when compared to wildtype HBZ (FIG. 1D). A novel TAX mutant was designed based on the CREB/ATF sequence with a tandem alanine mutation TAXΔ2. Hereafter, the mutant HBZAΔ27 will be referred to as HBZΔ1.

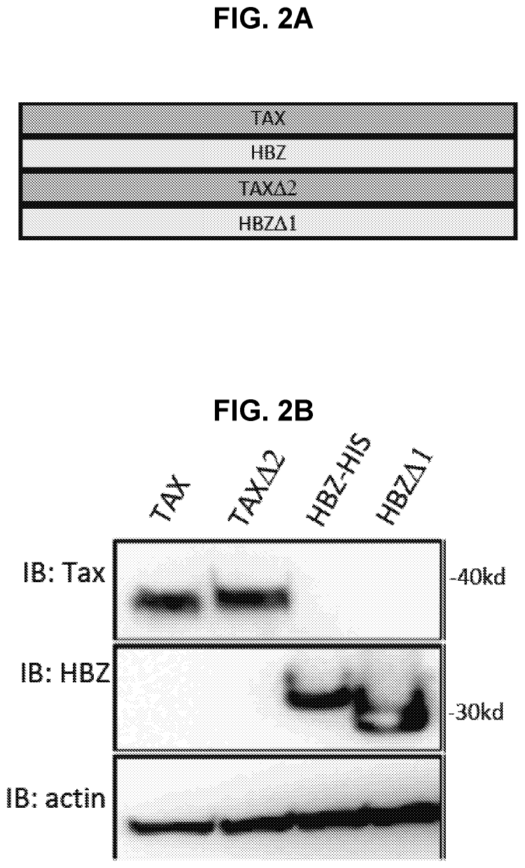

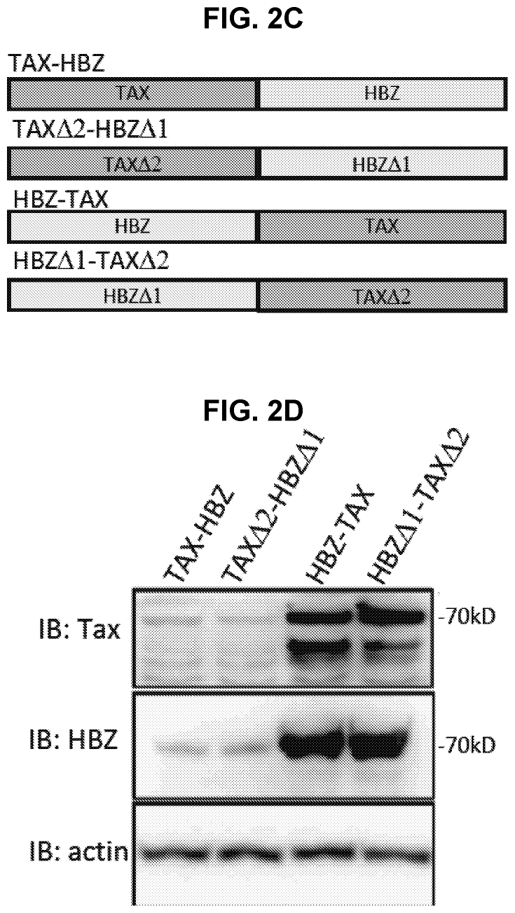

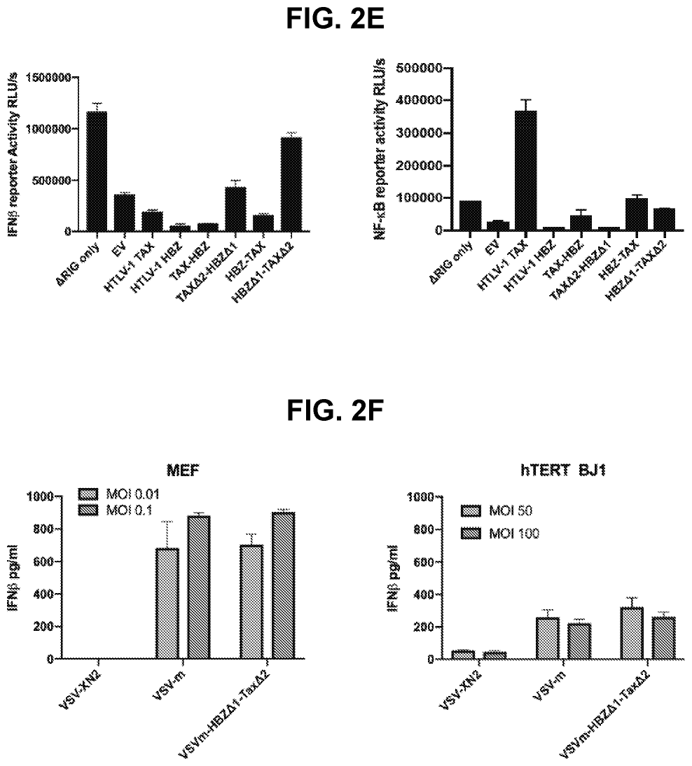

Based on the data from FIG. 1, novel mutations were created in TAX and HBZ using the tandem alanine approach (FIG. 2A). Each mutation was expressed to normal levels (FIG. 2B). Four novel fusion constructs were constructed based on the mutants designed from FIG. 1 (FIG. 2C). Each fusion construct has a fused HBZ-TAX polypeptide with either TAX or HBZ as the N terminal protein and the construct either encoded the wildtype or attenuated mutants of each protein (FIG. 2C). The TAX-HBZ protein expression levels were found to be far lower than the HBZ-TAX expression (FIG. 2D). Next, the fusion constructs were compared with their wildtype counterparts in additional reporter assays. The wildtype fusion significantly inhibited IFNβ and NF-kB reporter activity. The attenuated mutant fusions did not inhibit IFNβ promoter activity and decreased NF-kB, but to a lesser degree than the wildtype HBZ or wildtype HBZ-TAX fusion (FIG. 2E). Next, a VSVm construct was engineered to expresses the HBZΔ1-TAXΔ2 in the G-L transgene site. This VSVm construct has a triple alanine mutation in the M protein (52-54_{DTY-AAA}) that renders the matrix protein incapable of blocking host cell nuclear mRNA export allowing for a robust immune response. Inclusion of the HBZΔ1-TAXΔ2 was found to have no effect on the ability of infected MEF or hTERT cells (FIG. 2F) to produce IFNβ in response to VSV infection.

### Example 3: Construction and Characterization of VSV-gp62G-HBZΔ1-TAXΔ2

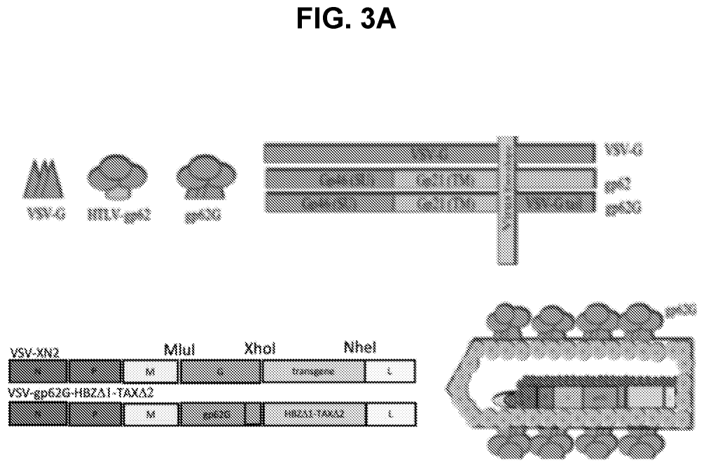

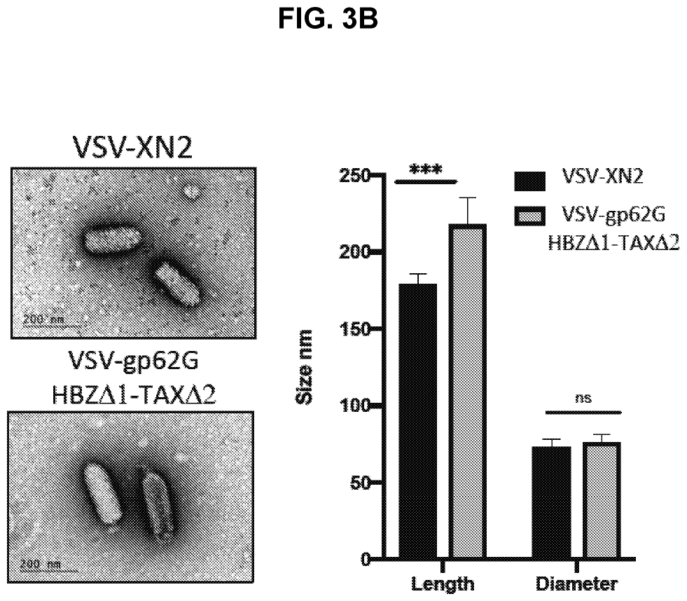

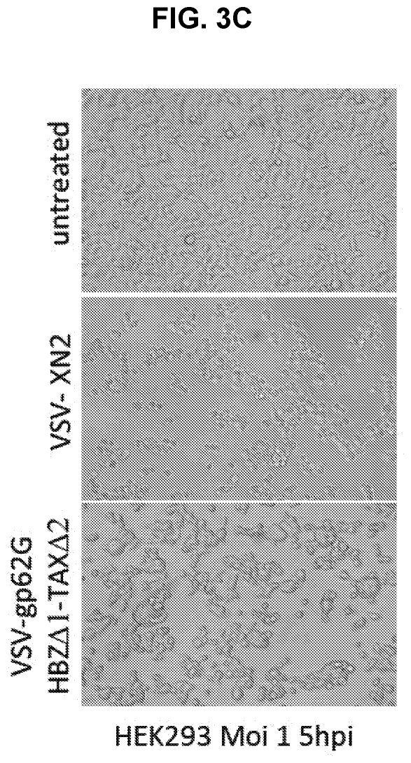

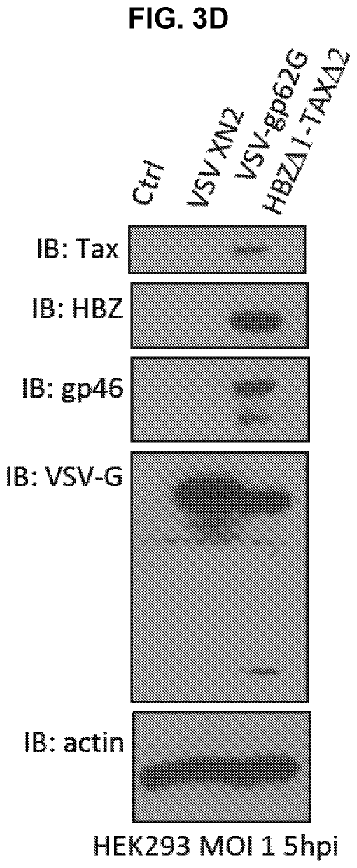

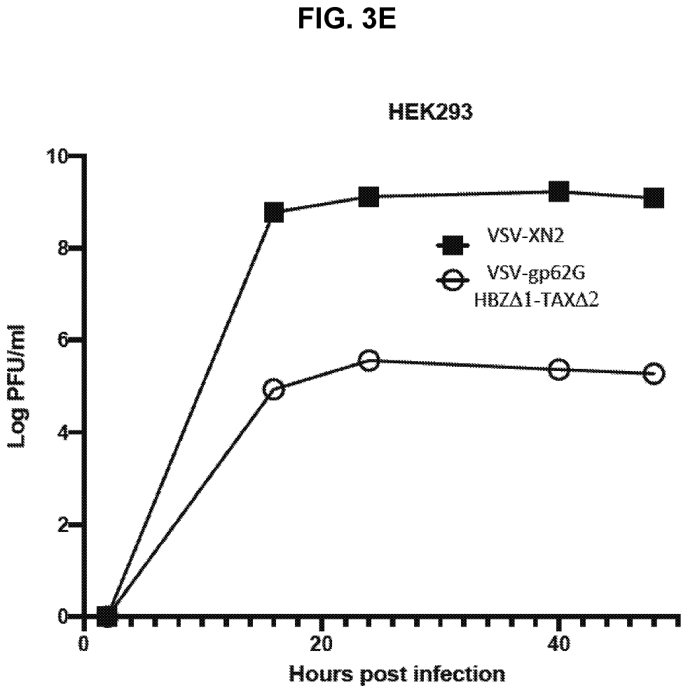

A VSV construct expressing a fusion of HTLV-1 env and VSV-G cytoplasmic tail was engineered (FIG. 3A). This construct had the VSV-G protein replaced with the gp62G, which should alter the tropism, growth kinetics and CPE of the virus. Additionally, the mutant HBZ-TAX (HBZΔ1-TAXΔ2) was encoded in the G-L transgene site of the virion. Analysis through transmission electron microscopy (TEM) indicated that the virions still retain the normal bullet-shaped morphology as VSV-XN2 although the virions were larger in length which was expected due to the helical genome organization in the virion and the large genome of VSV-gp62G-HBZΔ1-TAXΔ2 compared to VSV-XN2 (FIG. 3B). The VSV-gp62G-HBZΔ1-TAXΔ2 induced cellular fusion known as syncytia formation rather than the cell rounding effect typically found with VSV infection (FIG. 3C). Immunoblot analysis confirms that VSV-gp62G-HBZΔ1-TAXΔ2 expresses a fusion protein of approximately 70 kD which is the expected size of a HBZ (30 kD) and TAX (40 kD) fusion. This 70 kD protein is detected with both HBZ and TAX antibodies (FIG. 3D). Additionally, it was confirmed that VSV-gp62G-HBZΔ1-TAXΔ2 expresses a gp62G protein that retains a VSV-G tail that can be detected through our VSV-G antibody and a double band in the gp62 immunoblot that reflect the whole precursor protein and the gp46 subunit (FIG. 3D). Growth kinetic analysis in HEK293 cells at MOI 0.001 determined that VSV-gp62G growth was significantly attenuated (FIG. 3E).

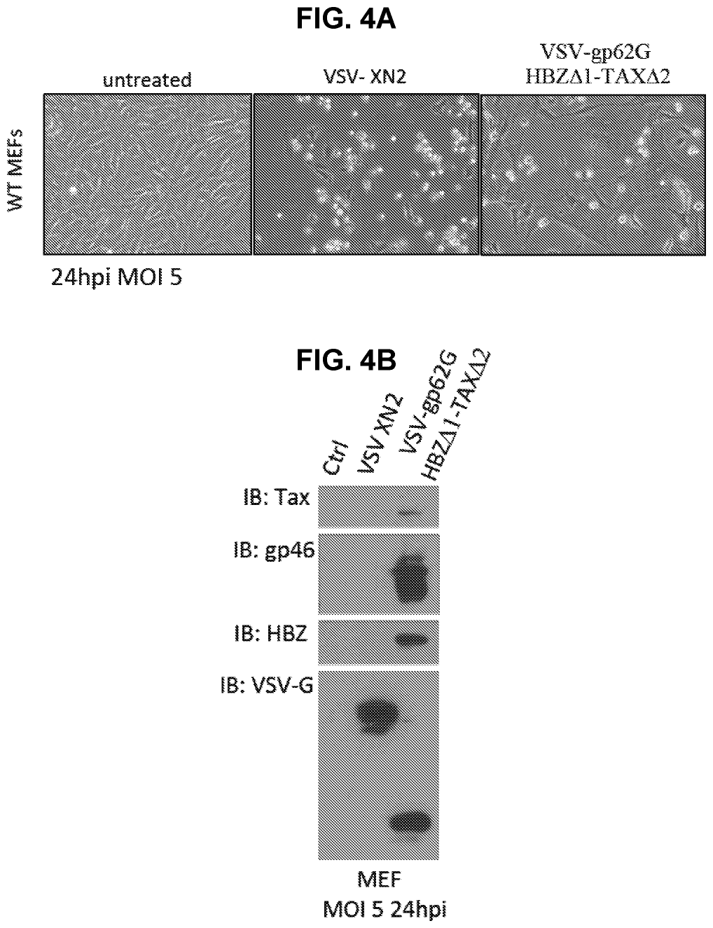

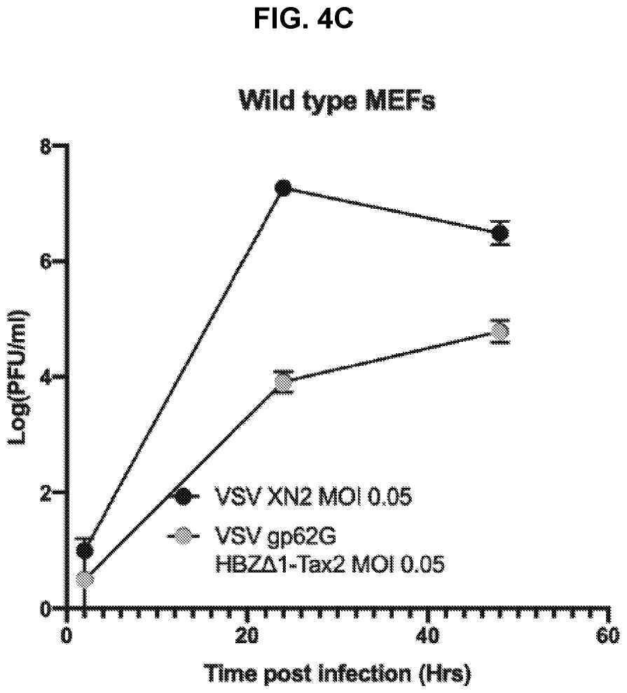

The VSV glycoprotein determines the tropism of the virus. The tropism of VSV was previously altered using a fusion gp160G, which conferred a tropism specific to hCD4+ CXCFR4+ cells. To determine if VSV-gp62G-HBZΔ1-TAXΔ2 is capable of infecting murine cells, murine embryonic fibroblasts, primary murine dendritic cells, and primary murine macrophages were exposed to VSV XN2 and VSV-gp62G-HBZΔ1-TAXΔ2 at MOI 5 and virus was measured in the supernatant at different time points. It was found that wildtype MEF were permissive to both VSV-XN2 and VSV-gp62G-HBZΔ1-TAXΔ2 (FIG. 4A). Immunoblot analysis at 24 hours post infection (hpi) at MOI 5 revealed expression of the viral proteins (FIG. 4B). VSV-gp62G-HBZΔ1-TAXΔ2 grew to significantly lower titers than VSV-XN2 (FIG. 4C). Furthermore, primary dendritic cells were permissive to VSV-XN2, but resistant to VSV-gp62G-HBZΔ1-TAXΔ2. Finally, primary macrophages were resistant to both VSV-XN2 and VSV-gp62G-HBZΔ1-TAXΔ2.

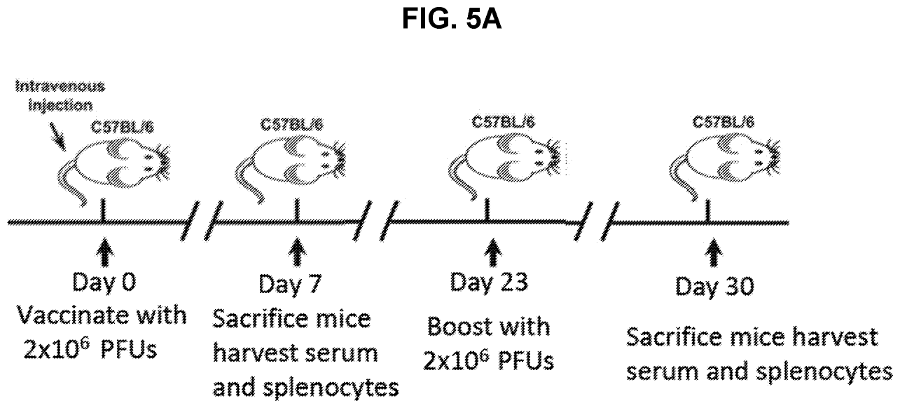

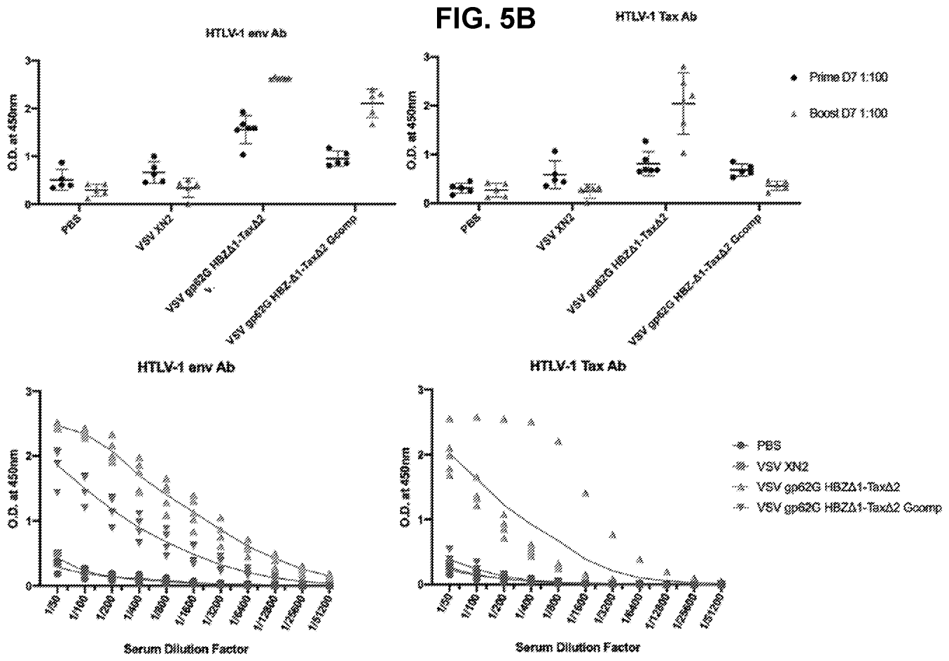

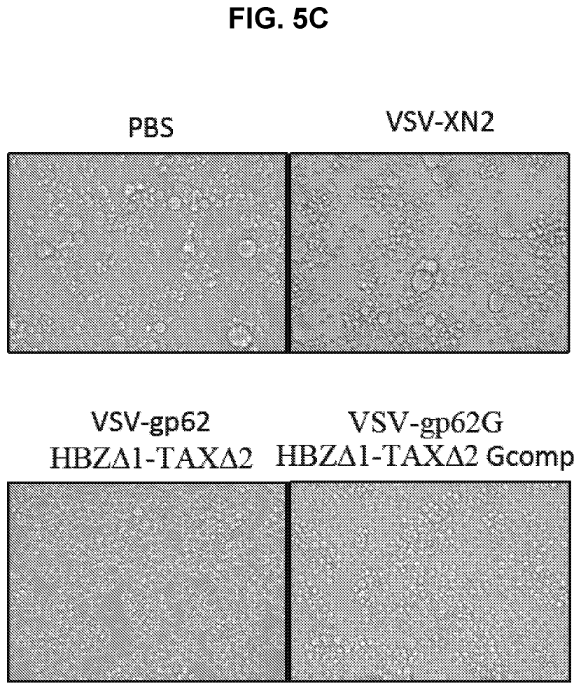

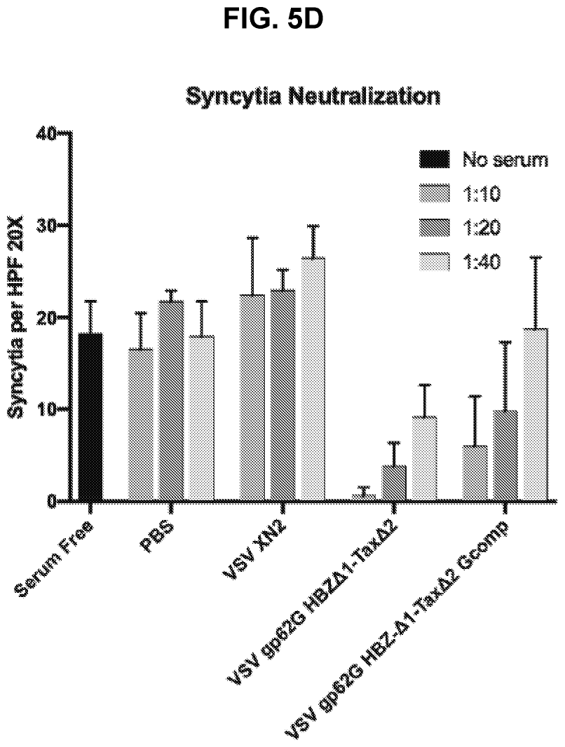

The vaccine efficacy of VSV-gp62G-HBZΔ1-TAXΔ2 was evaluated in a model using C57/BL6 mice. Mice were inoculated with 2×10⁶ PFUs of either VSV-XN2, VSV-gp62G-HBZΔ1-TAXΔ2, or VSV-gp62G-HBZΔ1-TAXΔ2 complemented with VSV-G in a prime boost strategy (n=10). Mice were inoculated on day 0 and day 23 and 5 mice were sacrificed on day 7 and the remaining 5 on day 30 (FIG. 5A). At the time of sacrifice, mice were anesthetized and exsanguinated via cardiac puncture and spleens were harvested to be analyzed for IFNγ secreting T cells through IFNγ ELISPOT and intracellular staining analyzed by flow cytometry. The serum was prepared from whole blood and analyzed for antibody titer against HTLV-1 gp62 and TAX. The VSV-gp62G-HBZΔ1-TAXΔ2 successfully induced an antibody response to both TAX and gp62. Additionally the response for both gp62 and TAX was significantly higher than the response from VSV-gp62G-HBZΔ1-TAXΔ2 animals complemented with VSV-G. Interestingly, the serum antibody levels for TAX actually decreased following boost, while VSV-gp62G-HBZΔ1-TAXΔ2 animals experienced a drastic rise in TAX antibody levels. Endpoint titration analysis of the boosted serum indicates that TAX antibodies reached cutoff at 1/6400 with one animal experiencing a cutoff exceeding 1/51200 dilution. For gp62 endpoint titration indicates that antibodies could be detected at cutoff exceeding 1/51200 dilution (FIG. 5B). Next, serum was analyzed for neutralizing activity via a fusion inhibition test using MT2 and K562 transfected with VCAM1. It was found that serum from VSV-gp62G-HBZΔ1-TAXΔ2 boosted animals significantly reduced syncytia formation at multiple dilutions tested (1:10 through 1:40) (FIGS. 5C and 5D).

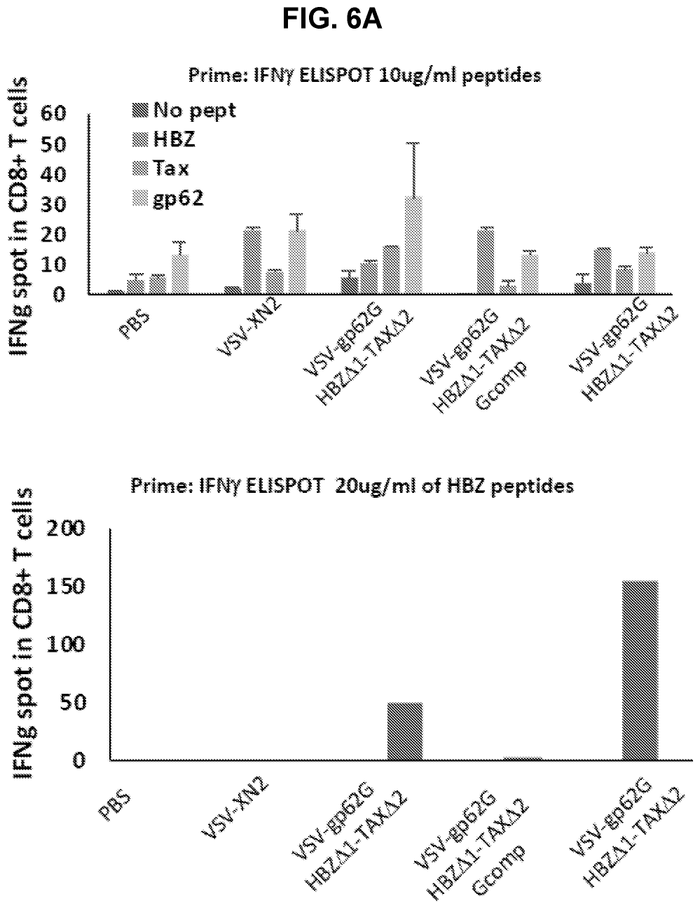

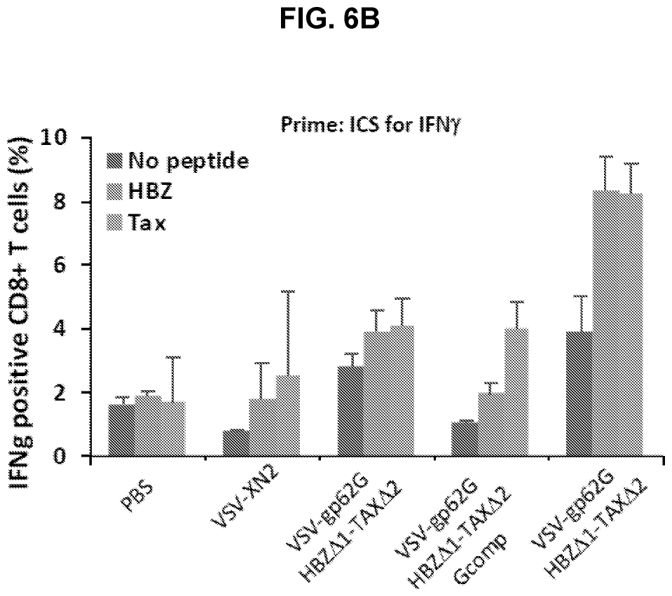

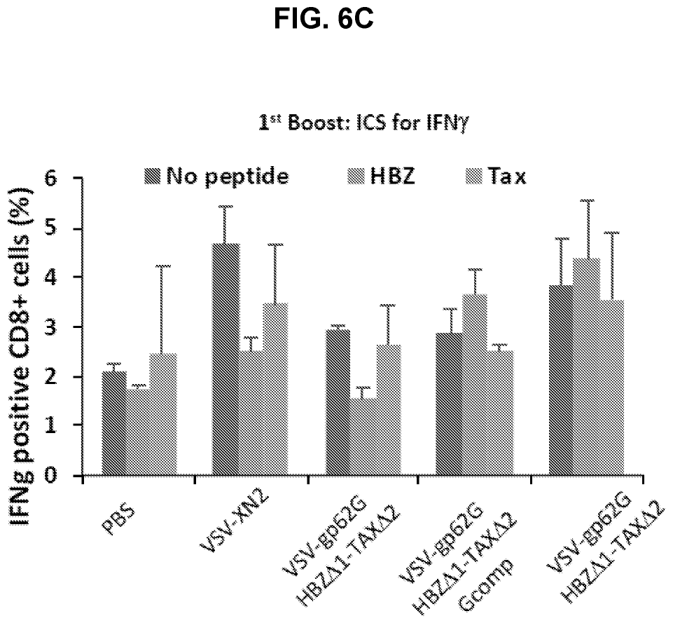

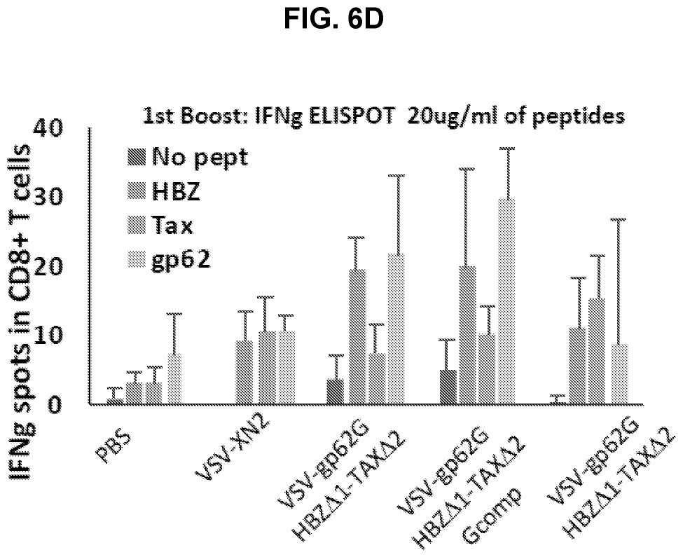

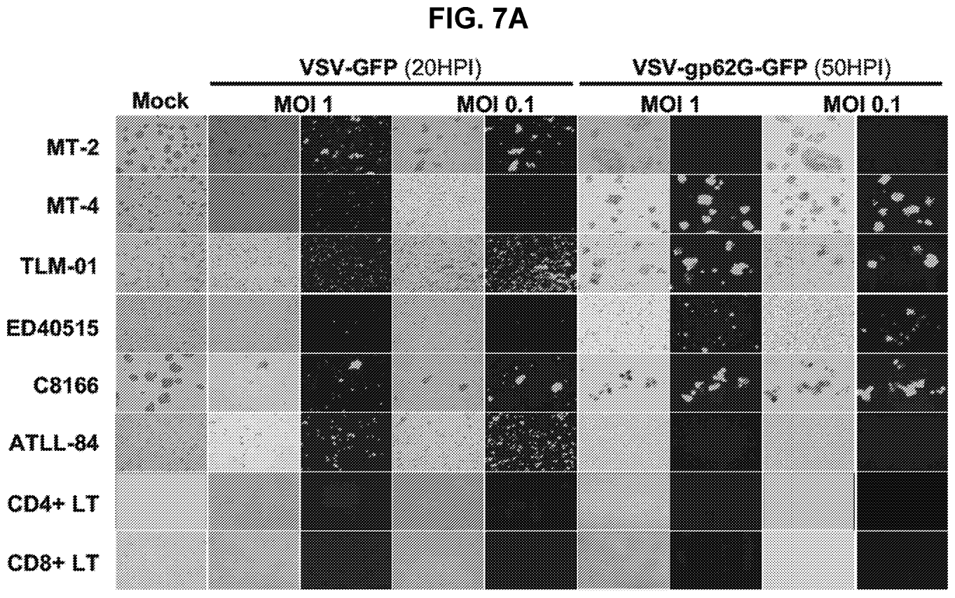

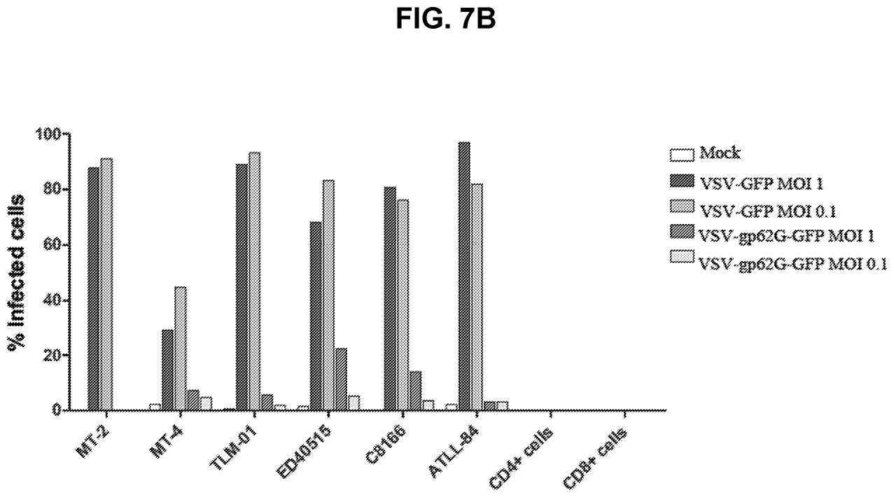

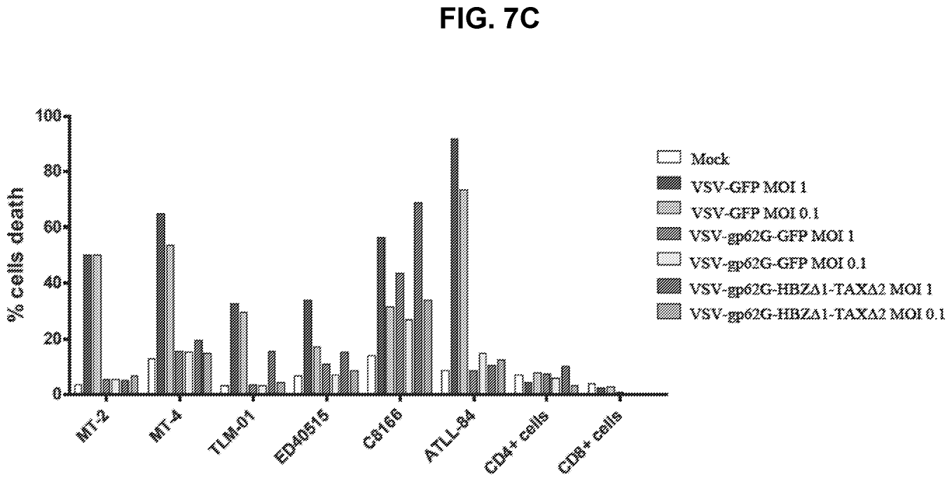

To assess the generation of a cell mediated CTL response, splenocytes from vaccinated animals were harvested and ELISPOT analysis from isolated CD8 T cells was performed to detect IFNγ secreting cells in response to peptide stimulation. A significant response to the HBZ peptide pool was detected at 20 ug/ml while peptide stimulation at 10 ug/ml did not produce significant results (FIGS. 6A and 6B). Intracellular staining for IFNγ was performed using isolated splenocytes and analysis cell by flow cytometry, and a significant response for HBZ and TAX peptide pool stimulation was observed (FIG. 6C). Animals were given a second inoculation of VSV, and splenocytes were harvested 7 days following the inoculation and analyzed for an immune response in a similar fashion following the initial inoculation. An increase in IFNγ secreting cells was detected from HBZ and gp62 stimulation, but a significant response for TAX peptides was not detected in the ELISPOT (FIG. 6D).

The oncolytic potential of VSV-gp62G-HBZΔ1-TAXΔ2 was also investigated. Several ATL lines were obtained and then infected with VSV-GFP and VSV-gp62G-GFP, and VSV-gp62G-HBZΔ1-TAXΔ2 at an MOI of 1 and 0.1. GFP expression was measured for the GFP expressing viruses and the percentage of cell death was calculated using a fixable viability dye. It was found that VSV-gp62G-GFP was able to infect MT4, TLM-01, ED40515, C8166 cells. Flow cytometry analysis also indicated that both VSV-gp62G-GFP and VSV-gp62G-HBZΔ1-TAXΔ2 were able to induce cell death.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

1-29. (canceled)

30. A vaccine, comprising:
a vesicular stomatitis virus (VSV) vector, where an engineered gene encoding a chimeric glycoprotein is substituted for a gene encoding a VSV glycoprotein G (VSV G), where the chimeric glycoprotein comprises an amino-terminal amino acid sequence from human T-cell leukemia virus type 1 gp62 protein and a carboxy-terminal amino acid sequence from the VSV G; and
an adjuvant.

31. A method of producing an immune response against human T-cell leukemia virus type 1 (HTLV-1), comprising administering to a subject in need thereof a vesicular stomatitis virus (VSV) vector, where an engineered gene encoding a chimeric glycoprotein is substituted for a gene encoding a VSV glycoprotein G (VSV G), where the chimeric glycoprotein comprises an amino-terminal amino acid sequence from human T-cell leukemia virus type 1 gp62 protein (HTLV-1 gp62) and a carboxy-terminal amino acid sequence from the VSV G.

32. The method of claim 31, where the VSV vector is administered with an adjuvant.

33. The method of claim 31, where the VSV vector is administered by intramuscular injection, subcutaneous injection, intradermal injection, oral administration, mucosal administration, or intranasal application.

34. The method of claim 31, where the subject is infected with HTLV-1.

35. The method of claim 31, where the subject was exposed HTLV-1.

36. The method of claim 31, where the subject is not infected with HTLV-1.

37. The method of claim 31, where the immune response comprises the subject generating antibodies to HTLV-1 gp62.

38. The method of claim 31, where the immune response comprises the subject generating antibodies to a human T-cell leukemia virus type 1 viral gene product TAX (HTLV-1 TAX).

39. The method of claim 31, where the immune response comprises the subject generating antibodies to a human T-cell leukemia virus type 1 basic lucine zipper factor (HTLV-1 HBZ).

40. The method of claim 31, where the immune response comprises the subject generating cytotoxic T cells to HTLV-1 gp62.

41. The method of claim 31, where the immune response comprises the subject generating cytotoxic T cells to a human T-cell leukemia virus type 1 viral gene product TAX (HTLV-1 TAX).

42. The method of claim 31, where the immune response comprises the subject generating cytotoxic T cells to a human T-cell leukemia virus type 1 basic lucine zipper factor (HTLV-1 HBZ).

43. A fusion protein comprising a vesicular stomatitis virus (VSV) vector, where an engineered gene encoding a chimeric glycoprotein is substituted for a gene encoding a VSV glycoprotein G (VSV G), where the chimeric glycoprotein comprises an amino-terminal amino acid sequence from human T-cell leukemia virus type 1 gp62 protein and a carboxy-terminal amino acid sequence from the VSV G, where the fusion protein further comprises:
a human T-cell leukemia virus type 1 basic lucine zipper factor (HTLV-1 HBZ);
a human T-cell leukemia virus type 1 viral gene product TAX (HTLV-1 TAX); and
at least 70-99% sequence identity to the amino acid sequence set forth in SEQ ID NO:2.

44. The fusion protein of claim 43, where the fusion protein further comprises at least 95% sequence identity to the amino acid sequence set forth in SEQ ID NO:18.

45. The fusion protein of claim 43, where the fusion protein further comprises at least 70-99% sequence identity to the amino acid sequence set forth in SEQ ID NO:6.

46. The fusion protein of claim 43, where the fusion protein further comprises at least 95% sequence identity to the amino acid sequence set forth in SEQ ID NO:20.

47. The fusion protein of claim 43, where the HTLV-1 HBZ is at the amino terminus of the fusion protein and the HTLV-1 TAX is at the carboxy terminus of the fusion protein.

48. The fusion protein of claim 43, where the fusion protein further comprises at least 70% sequence identity to the amino acid sequence set forth in SEQ ID NO:26.

49. The fusion protein of claim 43, where the fusion protein further comprises at least 95% sequence identity to the amino acid sequence set forth in SEQ ID NO:28.
